(19) United States
(12) Patent Application Publication
(10) Pub. No.: US 2023/0416799 A1
(43) Pub. Date: Dec. 28, 2023

(54) METHOD FOR INDUCING AND DETECTING SOLUBLE LOX-1 (sLOX-1) IN CULTURED BLOOD CLOTS

(71) Applicant: GEORGE MASON UNIVERSITY, Fairfax, VA (US)

(72) Inventors: Caroline Dieckmann Hoemann, Vienna, VA (US); Julia Alexa Leonard, Fairfax, VA (US); Virginia A. Espina, Rockville, MD (US); David Kepplinger, Fairfax, VA (US)

(73) Assignee: GEORGE MASON UNIVERSITY, Fairfax, VA (US)

(21) Appl. No.: 18/321,738

(22) Filed: May 22, 2023

Related U.S. Application Data
(60) Provisional application No. 63/344,258, filed on May 20, 2022.

(51) Int. Cl. C12P 21/02; G01N 33/86

(52) U.S. Cl. CPC C12P 21/02; G01N 33/86

(57) ABSTRACT

An embodiment of the invention provides a method of generating, ex vivo production of soluble Lox-1 (sLox-1), comprising: introducing a sample containing a blood free from an anti-coagulant factor into a device; adding a coagulation enhancing material in the sample; incubating the device; forming a cultured blood clot in the device; and shedding of the sLox-1 outside the cultured blood clot, wherein the method is configured to shed sLox-1 more than an anti-coagulated blood.

### CROSS-REFERENCE TO RELATED APPLICATION

This application claims the benefit under 35 U.S.C § 119 of U.S. Provisional Application 63/344,258 filed on 20 May 2022, titled as “METHOD FOR INDUCING AND DETECTING SOLUBLE LOX-1 (sLOX-1) IN CULTURED BLOOD CLOTS”, which is hereby incorporated by reference in its entirety.

### FIELD OF THE INVENTION

This invention relates to the process of production of sLox-1, more particularly to production of sLox-1 in the cultured blood clot, application of sLox-1 and the manner to produce sLox-1 in the cultured blood clot.

### BACKGROUND OF INVENTION

Lectin-like oxidized low-density lipoprotein receptor 1 (Lox-1) is a scavenger receptor that recognizes oxidized low-density lipoprotein (oxLDL) and activated platelets^{1,12}. In healthy arteries, Lox-1 expression is scarcely detected, but a chronic diet rich in saturated fats and cholesterol induces Lox-1 expression on coronary artery endothelial cells¹⁰. Endothelial cells exposed to oxLDL upregulate adhesion receptors that capture platelets, and monocytes that can become Lox-1-expressing macrophages, scavenge oxLDL and convert into “foamy” fat-laden cells^{12,13}.

Over time, these events contribute to stiffening of the arteries and the deposition of fatty plaques in the inner lining of the arteries. These structural changes contribute to hypertension which has a snowball effect of increasing vascular damage, ischemia, and cardiomyopathy. oxLDL also stimulates Lox-1-expressing cells to release pro-fibrotic mediators that drive tissue hypertrophy, fibrosis, atrial fibrillation¹⁴. Quivering of the atrium that occurs in diastolic or systolic dysfunction can lead to pooling of the blood and clot formation on the side-wall of the atrium. These clots can break free and lodge in the lung or brain tissue causing transient ischemia, strokes, and sudden death.

Although less-studied, cardiomyopathy can develop after severe trauma¹¹. A subset of Warfighters recovering from battlefield injuries may therefore face an additional long-term struggle with chronic heart disease. An understanding of why some individuals develop cardiomyopathy while sparing others is lacking.

Lox-1 has an established role in cardiovascular disease¹⁻¹⁰, and may have a role in trauma-induced cardiomyopathy¹¹.

Shock trauma after resuscitation from massive hemorrhage stimulates intravascular protease activity (ADAM17) that sheds the glycocalyx from endothelial cells leading to vascular injury¹⁵. These same proteases were shown to cleave the Lox-1 extracellular domain from this type II transmembrane receptor, to release soluble Lox-1 (sLox-1) (FIG. 1A).

sLox-1 was proposed as a biomarker of early-stage acute coronary syndrome and myocardial infarction^{17,18}, but the cells shedding the ectodomain have yet to be identified. In specific disease states (lung cancer, thrombotic influenza, thrombotic COVID), peripheral blood neutrophils can acquire Lox-1 expression¹⁹⁻²¹. In lung cancer patients, Lox-1 was identified as a biomarker of myeloid-derived suppressor cells (MDSCs) along with selected soluble mediators known to drive atrial hypertrophy and fibrosis (CXCL8, CXCL2, CCL2, IL1A, TNF, VEGFA)^{19,22}. A subset of these mediators were found in blood samples of patients with obstructive sleep apnea, a condition characterized by intermittent hypoxia/reoxygenation (IHR)^{8,9,23}.

These data suggest that Lox-1 and sLox-1 levels may be altered by hemorrhage, ischemia-reperfusion but in healthy human donors, endothelial cell expression of Lox-1 is scarce. The cells producing peripheral blood sLox-1 remain to be identified. It is currently unclear how to diagnose the risk or to treat trauma-induced cardiomyopathy. In patients with thrombocytopenia, is currently unclear how to diagnose low platelet counts caused by inadequate platelet production from low platelet counts caused by intravascular platelet activation (thrombosis).

### SUMMARY OF THE INVENTION

Methods for producing sLox-1 are currently limited to complex systems involving cultured cells, mainly bacterial cells that cannot properly fold, dimerize or glycosylate sLox-1. Autologous sLox-1 with personalized glycosylations is not currently available for clinical use.

Further, research papers till the time of the present invention could not identify the cell source of sLox-1 and assumed that the endothelial cells were shedding sLox-1 into the bloodstream.

In an embodiment, present invention provides a method of generating, ex vivo production of soluble Lox-1 (sLox-1), comprising: introducing a sample containing a blood free from an anti-coagulant factor into a device; adding a coagulation enhancing material in the blood; incubating the device; forming a cultured blood clot in the device; and shedding of the sLox-1 outside the cultured blood clot, wherein the method is configured to shed sLox-1 more than an anti-coagulated blood.

In an embodiment, the coagulating enhancing material comprises a lipopolysaccharide (LPS).

In an embodiment, the anti-coagulant factor comprises heparin, citrate, or ethylene diamine tetraacetic acid (EDTA).

In an embodiment, detecting one or more interleukins in the device.

In an embodiment, the one or more interleukins comprises IL-6, IL-8 and/or IL-18.

In an embodiment, addition of the inflammation enhancing material in the blood spikes shedding of sLox-1 into the device by about 20% to 60% more compared to a cultured blood clot free of the coagulation enhancing material.

In an embodiment, the method is configured to shed 0.5 ng to 50 ng of the sLox-1 per ml of the blood sample.

In an embodiment, the method is configured to produce an autologous sLox-1.

In an embodiment, the device is incubated at a temperature ranging from 20° C. to 40° C. for a time period ranging from 1 hour to 18 hours.

In an embodiment, the sLox-1 so sheds in the device is a personalized anti-coagulant or anti-thrombogenic agent.

In an embodiment, the device comprises a thrombus mimetic device.

In an embodiment, the method is configured to detect IL-8 and IL-6 concentrations in the cultured blood clot and the anti-coagulated blood.

In an embodiment, concentration of the sLox-1 in serum of the cultured clot compared to the anti-coagulated blood is from 0 pg/mL to about 50 ng/mL.

In an embodiment, concentration of the IL-8 in serum of the cultured clot compared to the anti-coagulated blood is from 50 pg/mL to 50 ng/ml.

In an embodiment, concentration of the IL-6 in serum of the cultured clot compared to the anti-coagulated blood is from 0 to 100 ng/mL.

In an embodiment, a device comprising a vacutainer tube and a means of heating to maintain temperature of the device at about 37° C., wherein the device is configured to screen agents that promote or inhibit one or more of the following: cell apoptosis, scramblase activity, flippase activity, ADAM17 activity, ADAM10 activity, alpha secretase activity, sLox-1 sheddase activity, tumor necrosis factor activation.

In an embodiment, the device is configured to screen a drug, antibody, nanoparticle, microbial-derived component, biomaterial, neutraceutical/dietary supplement as causing an increase or decrease in cultured clot serum sLox-1 relative to untreated cultured clot serum sLox-1, without inducing cell necrosis.

In an embodiment, the drug is a chemotherapeutic agent intended to induce apoptosis.

In an embodiment, the drug is configured to decrease ADAM17 activity and TNF activation.

In an embodiment, the drug is configured to suppress TNF expression or activity.

In an embodiment, the drug comprises a serine-threonine phosphatase inhibitor.

In an embodiment, the drug comprises beta glycerol phosphate.

### BRIEF DESCRIPTION OF THE DRAWINGS

The accompanying drawings, which are included to provide further understanding of the present invention disclosed in the present disclosure and are incorporated in and constitute a part of this specification, illustrate aspects of the present invention and together with the description serve to explain the principles of the present invention. In the drawings:

FIG. 1 shows a model showing how to produce sLox-1 in cultured clots and proposed therapeutic function of sLox 1 released by thrombi to diminish Lox 1 activation by endothelial cells and clot formation in blood vessels (A) protein structure, (B) OLR1 (Lox-1 mRNA) levels in (1) fresh blood (2) fresh blood clot (3): cultured blood clot (4 hr at 37° C.), (C) sLox-1 protein release to cultured clot serum, and (E) model proposing how thrombus-derived sLox-1 could protect endothelium from activated platelets and thrombin, in a donor-dependent manner depending on the amplitude of clot-induced sLox-1 shedding.

FIG. 2 shows significant induction of (A) OLR1 (Lox-1 mRNA) in cultured blood clots and LPS/cultured clot and (B) sLox-1 shedding into cultured clot serum and LPS/cultured clot serum relative to fresh whole blood, fresh clot (45 min RT) and heparin blood. Numbers 1-6 in the figure on the X-axis refers to: 1) fresh blood plasma, 2) 45 minute RT clot, 3) cultured clot (4 h, 37° C.), 4) lipopolysaccharide/cultured clot (4 h, 37° C.), 5) bGP/cultured clot (4 h, 37° C.), and 6) cultured heparin blood (4 h, 37° C.).

FIG. 3. TEG assay of recalcified citrated plasma (260 μL) combined with 100 μL of (A) fresh clot serum (45 min RT) or (B) cultured clot serum (4 hours at 37 C) from the same donor. Cultured clot serum showed reduced thrombogenic activity relative to fresh serum. Y-axis: mm (clot tensile strength). X-axis: time (minutes). The white trace shows the amplitude of the clot tensile strength. The gray trace shows velocity of coagulation. This figure shows cultured clot serum has lower thrombogenic activity compared to fresh clot serum. Thromboelastography assay of pooled human citrated plasma (160 μL) combined with either (160 μL) fresh clot serum (A) or cultured clot serum (B) from the same donor, and then submitted to the TEG test with 20 μL of 200 mM CaCl₂. The clotting time, clot velocity, and maximal amplitude reflecting clot tensile strength were around 2-fold less for cultured clot serum that contains 460 pg/mL of sLox-1 compared to fresh serum which had 0 pg/mL sLox-1.

FIG. 4 shows a cultured clot device according to an embodiment of the invention.

FIG. 5A shows high quality total RNA from fresh (anticoagulated) blood (FB) and cultured blood clot samples Polytron-homogenized into PAXgene buffer and lower quality total RNA from (condition 9) cultured clot samples vortex-mixed into PAXgene buffer.

FIG. 5B shows the yield of total RNA from fresh blood (FB) and cultured blood clot samples Polytron-homogenized into PAXgene buffer or (9) vortex-mixed into Paxgene buffer.

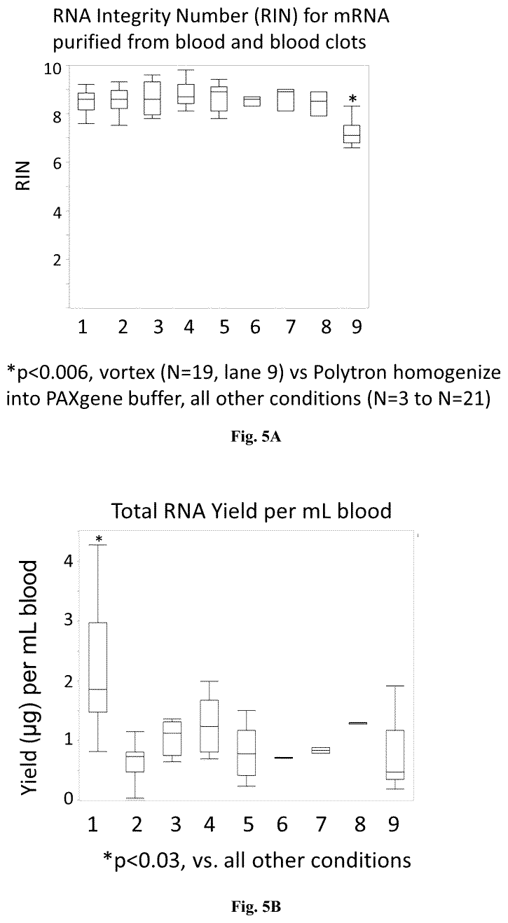

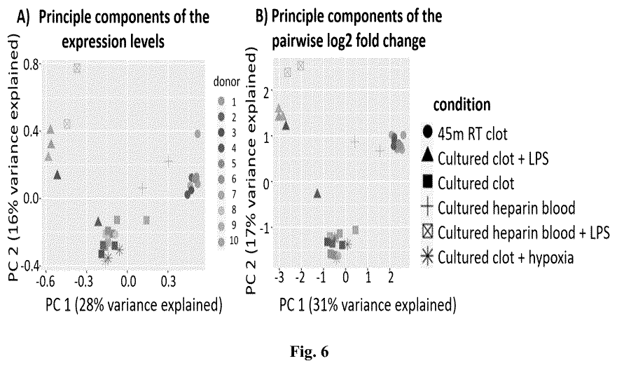

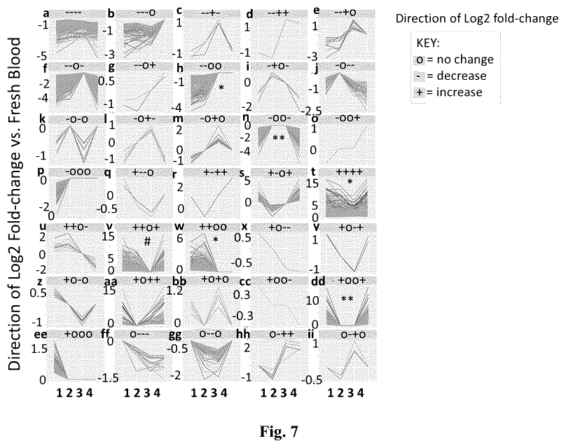

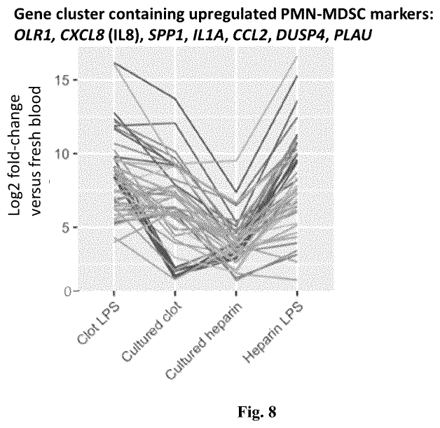

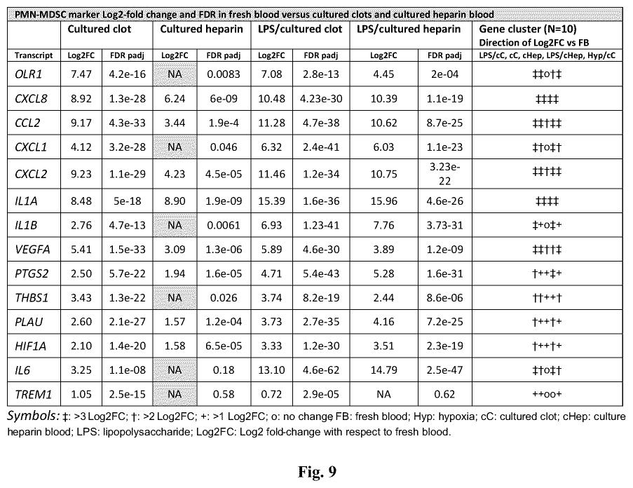

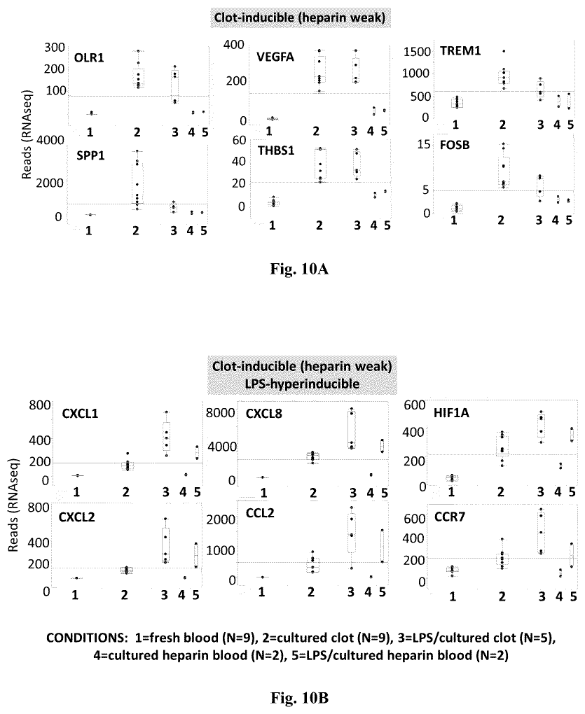

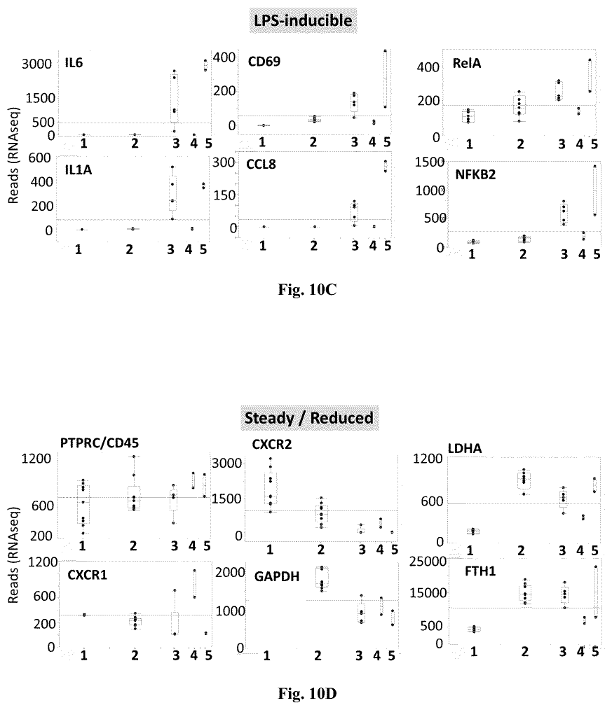

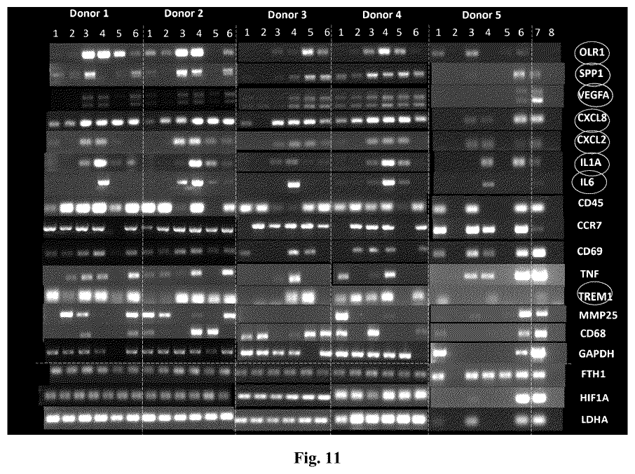

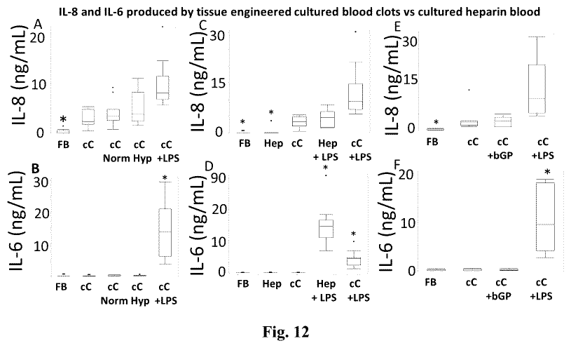

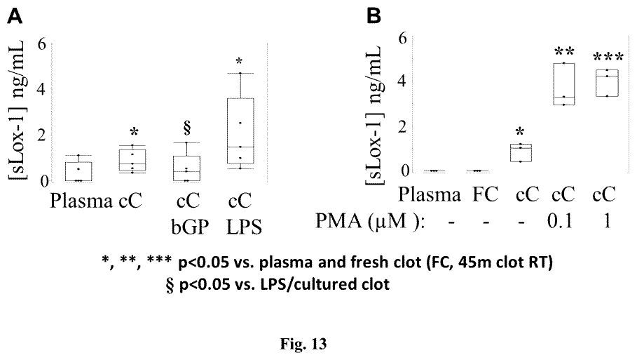

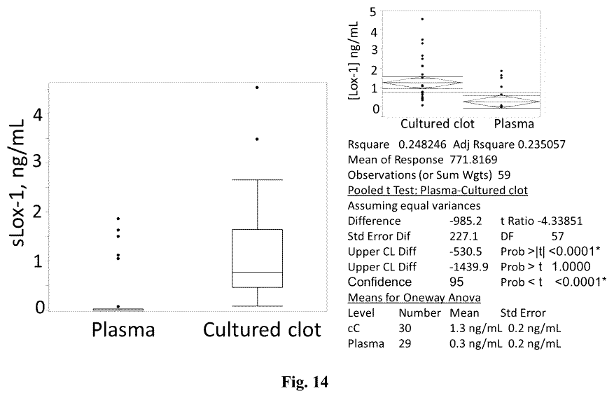

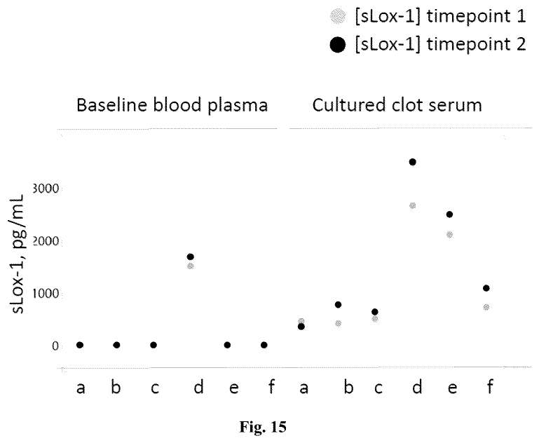

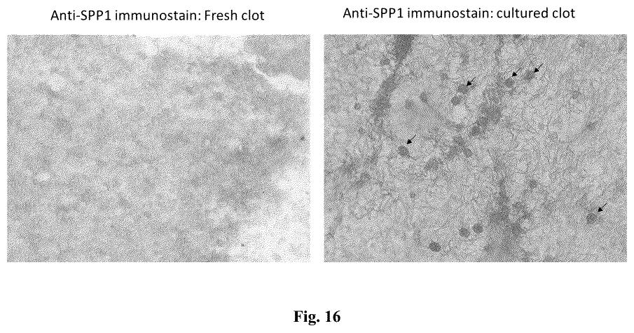

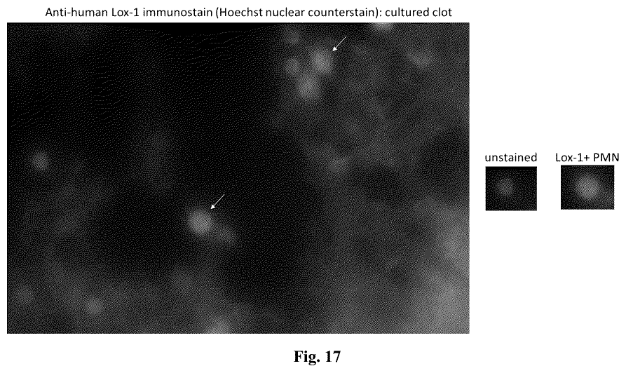

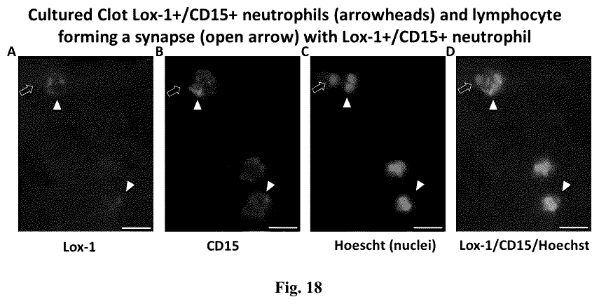

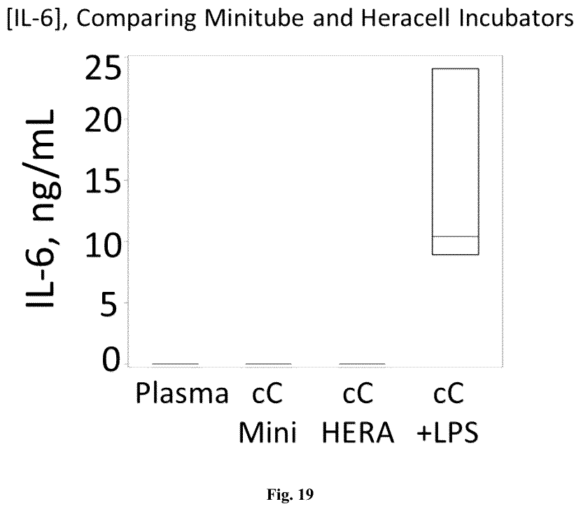

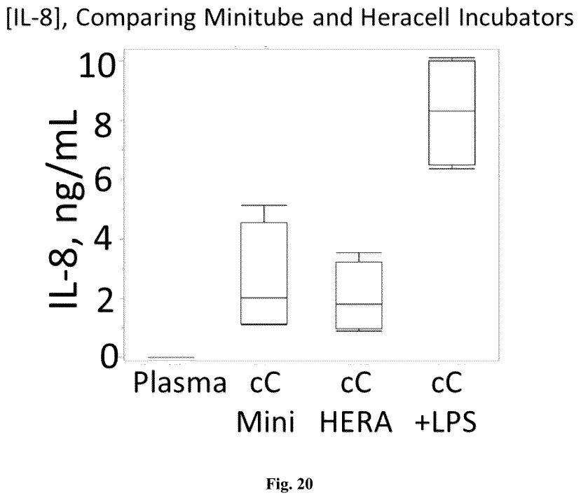

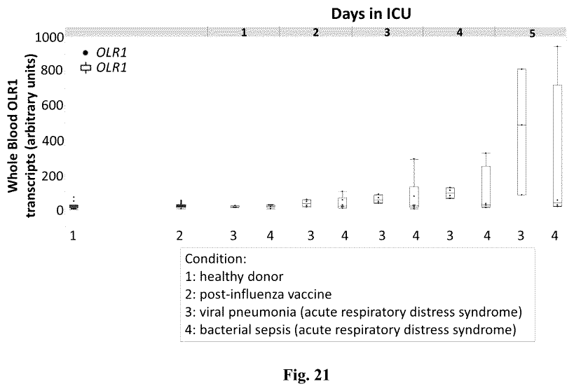

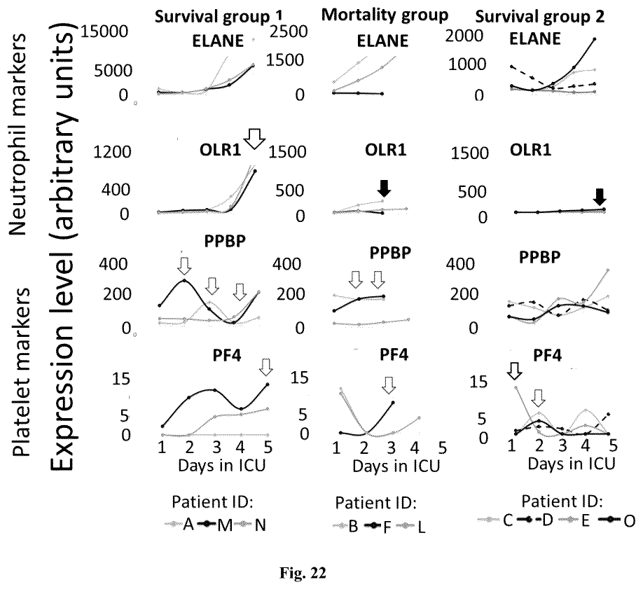

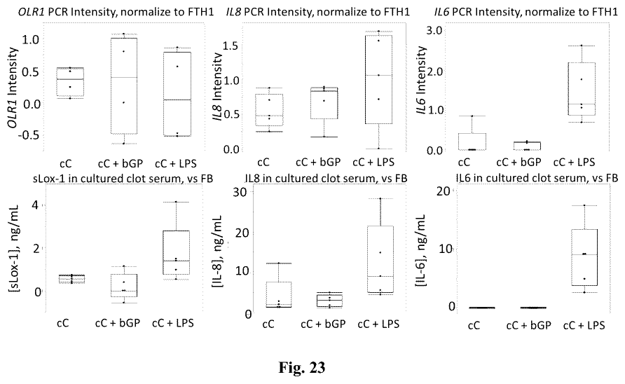

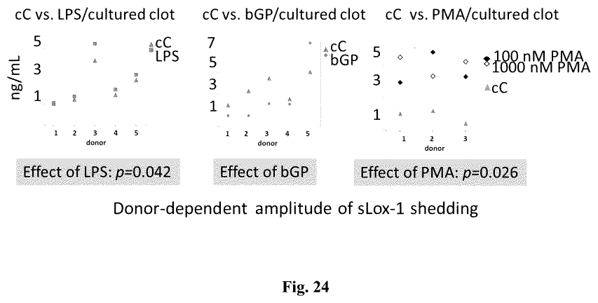

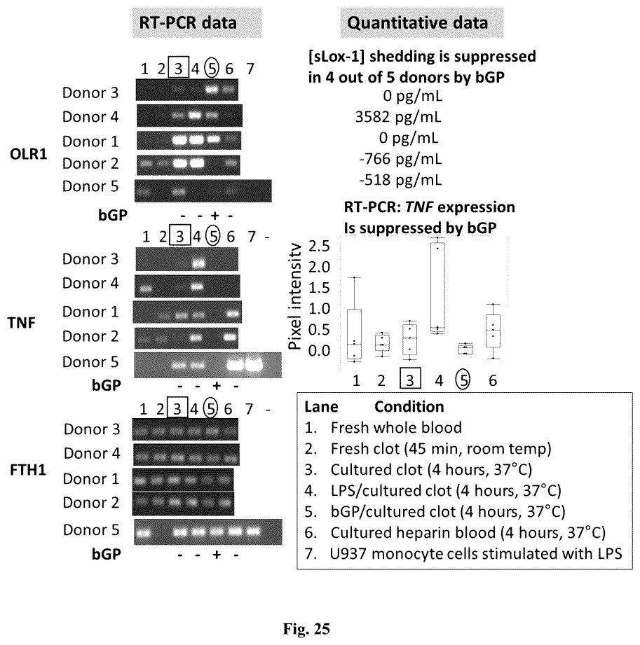

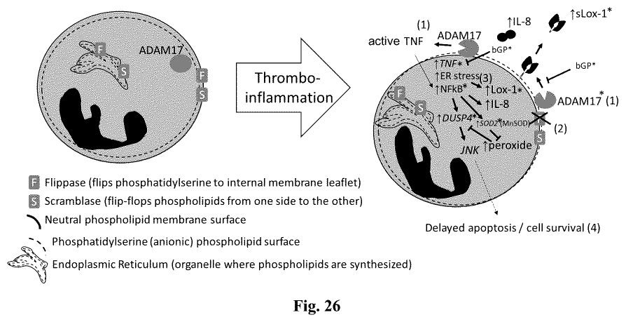

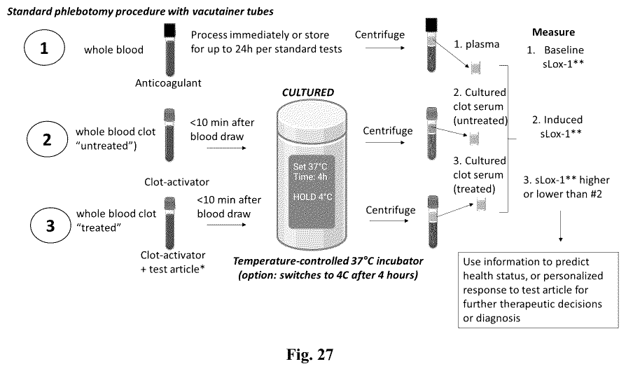

In FIGS. 5A and 5B, ANOVA shows cultured clot vortex mixed into PAXgene reagent has significantly lower RNA integrity number (RIN) than all other conditions, p<0.006. FB has significantly higher RNA yield than all other conditions, p value p<0.03. FB N=21, RV, red vacutainer tube cultured blood clot N=17, Normoxia cultured blood clot N=9, Hypoxia cultured blood clot N=7, RV treated with LPS N=15, Heparin (fresh anticoagulated blood) N=3, Cultured Heparin Blood N=3, Heparin-LPS: Cultured Heparin blood treated with LPS N=3, CC vortex (cultured clot vortex mixed into PAXgene) N=19.

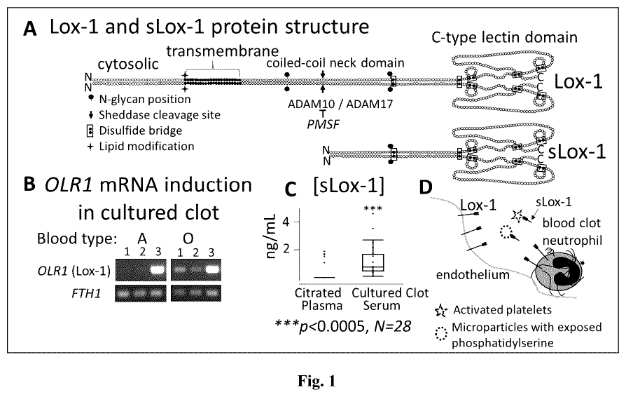

In FIGS. 5A and 5B, 1-9 on the X-axis refers to: 1) fresh blood plasma 2) cultured clot red vacutainer (RV) tube, 3) cultured clot, glass tube, Normoxia, 4) cultured clot, glass tube, Hypoxia, cultured clot+LPS, 6) heparin blood, 7) cultured heparin blood, 8) cultured heparin blood+LPS, and 9) represents cultured clot, vortex mix into PAXgene.

FIG. 6 provides scores on the first two principal components (PCs) of the (A) expression levels and (B) the scores on the first two PCs of the pairwise log 2 fold changes. Healthy non-fasting and consenting volunteers provided blood samples to analyze fresh blood (N=10), cultured clots (4 h at 37° C., N=10), cultured clots with added 100 ng/mL lipopolysaccharide (4 h at 37° C., LPS, N=5), cultured heparin blood with and without 100 ng/mL LPS (4 h at 37° C., N=2). PCA analyses showed that cultured clot global gene expression varied from that of fresh blood and LPS-stimulated samples. In one donor out of 10, the fresh blood profile clustered with cultured clot samples. LPS stimulated a distinct response in 4 out of 5 donors from cultured clots according to PCA components of the expression levels.

FIG. 7 provides cluster analysis of differentially expressed genes vs fresh blood according to the direction of Log2 fold-change in gene expression, in (left to right): LPS/cultured clots (n=5), cultured clots (n=10), cultured heparin (n=2), LPS/cultured heparin (n=2). Symbols illustrate gene clusters identified as upregulated or downregulated in cultured clot (*) refers to cultured clot-specific response genes (panels h, t, w)), (#) refers to cultured clot and LPS-induced genes (panel v), and (**) refers to LPS-specific response genes (panels n, dd).

Numbers (1-4) on x-axis refers to: 1) cultured clot+LPS, 2) cultured clot, 3) cultured heparin blood, and 4) cultured heparin blood+LPS. Number of genes in each cluster: a) 378, b) 95, c) 3, d) 1, e) 18, f) 912, g) 2, h) 587, i) 3, j) 50, k) 10, l) 5, m) 11, n) 356, o) 1, p) 190, q) 2, r) 1, s) 69, t) 594, u) 11, v) 757, w) 549, x) 1, y) 2, z) 9, aa) 56, bb) 5, cc) 1, dd) 486, ee) 128, ff) 16, gg) 80, hh) 6, ii) 3.

FIG. 8 provides log 2 fold-change (LFC) for cultured clot+LPS, cultured clot, cultured heparin and cultured heparin+LPS. The cluster included OLR1 (mRNA encoding Lox 1) and genes previously identified by Veglia et al (2021) Nature Reviews Immunology 21:485 498, as being activated in human myeloid derived suppressor cells (CXCL8/IL8, SPP1, IL1A, CCL2, DUSP4, PLAU). Following genes are included in this cluster: AK4, F3, RGS1, IL10, LAMB3, GOS2, BANAM2-AS1, IL1RL2, IL1A, IL1B, NR4A2, CCL20, HRH1, PPRAG, CXCL8, CXCL3, CXCL2, EREG, AREG, SPP1, HBEGF, C6orf223, HILPDA, CLEC5A, EGR3, DUSP4, NR4A3, TNFSF15, PLAU, OLR1, GPR84, GJB2, C15orf48, CCL22, CCl2, CCl7, CCl3, KCNH4, COL1A1, SNAI1, SLCO4A1, LIF, OSM, CCL3_1, CCL3L3_1, CCL3L3_2, CCL4L2_2, CCL3L1, CCL4L1.

FIG. 9 shows OLR1 (Lox-1 mRNA) is unregulated in cultured clots (ORL1: LFC 7.47 in cultured clot, False Discovery Rate padj=4.2e-16) along with MDSC signature genes and in blood cultures in heparin vacutainer tubes to a much lesser degree.

FIGS. 10A, 10B, 10C, 10D show fresh blood is devoid of markers of PMN MDSCs in 9 out of 10 healthy donors whereas all cultured blood clots develop expression of markers previously associated with PMN MDSCs including OLR1 (Lox 1), CXCL8, IL6, CD69, CXCL1, VEGFA, TREM1, CXCL2, THBS1, and IL1A (figure shows N=9 healthy human donors). Differences between fresh blood (1), cultured clot (2), LPS/cultured clot (3), cultured heparin blood (4), LPS/cultured heparin blood (5) samples were analyzed according to TPM read counts by RNAseq (line: grand mean; mean diamonds: mean and quartiles; points). Pooled t Test showed significant differences of the mean comparing cultured clot with fresh blood with p≤0.001 for all markers except for IL6 (p<0.05) and IL1A (p<0.01) versus cultured clot no LPS. Gene expression profiles were categorized as being induced by clot culture, by clot culture and more induced by LPS, only induced by LPS, or highly expressed/reduced. OLR1 expression levels correlated with FosB levels (i.e. AP-1, R\hat{ }2=0.75, p<0.0001) but not with RelA expression levels. [Conditions: 1=fresh blood (N=9), 2=cultured clot (N=9), 3=LPS/cultured clot (N=5), 4=cultured heparin blood (N=2), 5=LPS/cultured heparin blood (N=2).

FIG. 11 shows RT-PCR validation of RNA sequencing experiment shows that OLR1 and MDSC markers are induced in cultured clots relative to fresh blood and fresh blood clots in blood samples from N=5 non-fasting healthy consenting male and female donors with blood types A or O; Lewis Leb, Lea or null; Fya/Fyb, Duffy Fya, Fyb, or Fya/Fyb, secretor and nonsecretor. Lanes: 1. Fresh whole blood (PaxGene); 2. Fresh blood clot (45 min, RT); 3. Cultured clot (4 h, 37° C.); 4. LPS/cultured clot (4 h, 37° C.); 5. Beta-glycerol phosphate cultured clot (4 h, 37° C.); 6. Cultured heparin blood (4 h, 37° C.). OLR1 was induced in all cultured clots (4) and in all cultured clots+LPS (lane 5). IL6 was only induced by LPS (lane 4). TNF was specifically suppressed in cultured clots treated with 10 mM disodium beta glycerolphosphate (lane 5), without suppressing expression of CXCL8/IL-8, or housekeeping genes FTH1 and LDHA.

FIG. 12 shows Compared to blood plasma and fresh clot serum, cultured clot serum shows elevated CXCL8/IL8 but not IL-6. Cultured LPS/clot serum shows elevated CXCL8/IL-8 and IL-6. Adding beta glycerol phosphate to the clot does not alter clot induced CXCL8/IL-8. [Key: FB: fresh blood plasma, cC: cultured clot (4 h, 37° C.), Norm: cultured clot under Normoxia, Hyp: cultured clot under Hypoxia, Hep: heparin blood culture (4 h, 37° C.), +LPS: +100 ng/mL lipopolysaccharide, +bGP: +10 mM disodium beta glycerol phosphate].

FIG. 13 shows sLox-1 serum levels, for the samples shown in FIG. 12, panels E and F, and PMA-treated cultured clots. This is the enabling data for beta glycerol phosphate and PMA treated blood clots. Beta glycerol phosphate suppressed sLox-1 shedding and PMA enhanced sLox-1 shedding into cultured clot serum. bGP only significantly lower than cC+LPS.

FIG. 14 shows OLR1 gene expression in cultured blood clots leads to sLox-1 production in cultured clot serum (ELISA assay, N=21 biological replicates and N=7 technical replicates). ELISA shows that sLox-1 is induced and released to the serum of cultured clots, analysis of n=28 donors with n=21 biological replicates and n=7 technical replicates. Technical replicates were reproducible over time and distinct blood draws. sLox-1 was elevated in baseline plasma 4 out of 21 healthy donors (blood type A or type O), and in these donors, sLox-1 was more significantly induced in cultured clot serum (t ratio=−4.38 assuming unequal variances, p<0.0001; 95CI: −53.4 to 595 pg/mL plasma, 937 to 1574.9 pg/mL cultured clot).

FIG. 15 shows precision medicine: Donor specific sLox 1 in plasma and sLox 1 generation in cultured clot serum. [sLox1] pg/mL for 6 human donors (a to f).

FIG. 16 shows SPP1 expressing mononuclear cells are detected in cultured clots (dark brown signal, HRP substrate). SPP1 is considered a biomarker of PMN-MDSCs.

FIG. 17 shows anti-human Lox-1 immunostain: cultured clot unstained. Lox-1 expressing mononuclear cells and polymorphonuclear cells are detected in cultured clots (white arrow, grey signal, ABC AP red substrate, epifluorescence microscopy, DAPI counterstained nuclei).

FIG. 18 shows cultured Clot Lox-1+/CD15+ neutrophils (arrowhead) and lymphocycte forming a synapse (open arrow) with Lox-1/CD15+ neutrophil.

FIGS. 19 and 20 show cultured clots can be produced using any incubator or condition that maintains the clot around 37° C. In this example, vacutainer tubes were placed in a humidified cell culture incubator (Heracell) or in a Minitube® portable 37° C. incubator. [cC=Cultured clot].

FIG. 21 shows Averaged whole blood OLR1 transcript levels in whole blood RNA from patients with sepsis or viral pneumonia induced Acute Respiratory Distress Syndrome (ARDS). The averaged data reveal an effect of day, but do not reveal an effect of disease condition or disease severity. (meta-analysis of data from Parnell et al, Shock, 2013; 40:166-174). [conditions: 1: Healthy donor, 2: post-influenza vaccine, 3: viral pneumonia (acute respiratory distress syndrome), 4: bacterial sepsis (acute respiratory distress syndrome)].

FIG. 22 shows analysis of personalized regulation of OLR1 in whole blood transcript levels in patients with Acute Respiratory Distress Syndrome, during 6 days of ICU oxygen ventilation suggests a potential thromboprotective effect of OLR1 expression. Bacterial sepsis (A, B, C, D, E), viral pneumonia (L, M, N, O): OLR1 levels rose in patients A, M, N following or concomitant with platelet markers. ELANE: neutrophil elastase; OLR1: oxidized LDL receptor 1 (PMN-MDSC marker) PPBP: pro-platelet basic protein; PF4: platelet factor 4; Patient condition after day 6 was not reported. Metadata analysis of RNAseq data from samples published by Parnell et al. Identifying Key Regulatory Genes in the Whole Blood of Septic Patients to Monitor Underlying Immune Dysfunctions. Shock; 2013; 40:166-174.

FIG. 23 shows RT-PCR of transcript levels (OLR1, IL8, IL6) in cultured clots from healthy donors, and matching soluble factor released to cultured clot serum, corrected to fresh blood. Averaged data show that OLR1 is induced in the cultured clot and that sLox-1 shedding to the serum is increased by LPS and inhibited by a phosphatase inhibitor. Parallel analysis of IL-8/CXCL8 in the cultured clot serum can be used to document test article cytocompatibility. The absence of IL6 in donor fresh blood can be used to verify there is no sepsis or bacteremia, and lack of IL-6 induction in the cultured clot serum can be used to identify sterile inflammation and test article purity (lack of endotoxin response).

FIG. 24 shows personalized regulation of sLox-1 sheddase activity in cultured blood clots from healthy donors by endotoxin or by a drug (phosphatase inhibitor, beta glycerol phosphate, bGP, or phorbolmyristate acetate, PMA). These data show that the cultured clot device could be used as a precision medicine device to test for drug-induced responses that aim to reduce or increase the abundance of cells expressing Lox-1 (i.e., PMN-MDSCs), or alter sLox-1, or mediators upstream of sLox-1 shedding, including ADAM17 sheddase activity, phosphatidylserine exposure which is needed for ADAM17 activation, apoptosis which causes phosphatidylserine exposure, or TNF activity which depends on ADAM17. ADAM17 is also called TNF activating convertase enzyme, TACE.

FIG. 25 shows RT-PCR data for OLR1, FTH1, TNF from different types of clots. “-” identify the negative control lane (no cDNA template).

FIG. 26 shows model of sLox-1 shedding as a biomarker of upstream/parallel cellular activities. 1) Sommer et al. Nature Comm. 2016; 7: 11523. 2) Sakuragi et al. PNAS 2019. 116 (8) 2907-2912; 3) Condamine et al. Science Immunology 2016 August; 1(2); 4) Kamata et al. 2005. Cell 120: 649-661.

FIG. 27 shows standard phlebotomy procedure with vacutainer tubes.

### DETAILED DESCRIPTION OF THE INVENTION

### Definitions and General Techniques

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

No element, act, or instruction used herein should be construed as critical or essential unless explicitly described as such. Also, as used herein, the articles “a” and “an” are intended to include items, and may be used interchangeably with “one or more.” Furthermore, as used herein, the term “set” is intended to include items (e.g., related items, unrelated items, a combination of related items, and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, health monitoring described herein are those well-known and commonly used in the art.

The methods and techniques of the present invention are generally performed according to conventional methods well known in the art and as described in various general and more specific references that are cited and discussed throughout the present specification unless otherwise indicated. The nomenclatures used in connection with, and the procedures and techniques of embodiments herein, and other related fields described herein are those well-known and commonly used in the art.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The percentage given any should be construed based on the weight %, unless indicated otherwise.

The following terms and phrases, unless otherwise indicated, shall be understood to have the following meanings.

As used herein, the terms “subject”, “patient” and “subject in need thereof” may be used interchangeably and refer to a subject in need of administration of the pharmaceutical composition of the invention, or in need of pre-operative, post-operative, or periodic blood sampling to document health status. The term “subject” denotes a mammal, such as a rodent, a feline, a canine, an equine, a goat, a pig, a transgenic pig, a bovine, and a primate.

As used herein, the term “ex vivo” refers to the process by which cells are removed from a living organism and grown outside the organism (eg, a test tube). As used herein, the term “in vitro” refers to the process by which cells known to grow only in vitro (eg, various cell lines, etc.) are cultured.

Ex vivo production of sLox-1, according to this aspect of the present invention, provides white blood cells (WBC) with conditions for cell growth ex vivo, culturing WBC ex vivo with or without cytokine, thereby allowing production of sLox-1. In another aspect of the present invention, Ex vivo provides neutrophils with conditions for cell growth, culturing neutrophils ex vivo with or without cytokine, thereby allowing production of sLox-1.

As used herein, “culturing” provides the chemical and physical conditions (eg, temperature, gas) and growth factors required for the maintenance of blood clot. In an embodiment, culturing provides the chemical and physical conditions for maintenance of neutrophils.

As used herein, “cultured blood clot” or “cultured clot” refers to a process when the blood is allowed to clot, for example: a small volume of blood is collected in a sterile container and allowed to clot within an effective duration time to form a clot. In an embodiment, the clot could be collected and put into in a suitable broth to collect serum.

In an embodiment, “cultured clot” is different from “fresh blood clot.” The fresh blood clot is formed by incubation at room temperature for at least 45 minutes and up to as long as standard laboratory tests for patient serum allow.

The advantage of clot culture is that serum can be collected and investigated for serological tests. The cultured blood clot could be either treated or untreated.

As used herein, term “treated cultured blood clot” or “treated cultured clot” refers to when the formation of the blood clot is positively influenced by addition of a drug, inflammatory factor, enhancing factor, coagulant factor, antibody, nanomaterial, nucleic acid, enzyme, pathogen-derived factor etc.

As used herein term, “untreated cultured blood clot” refers to when the formation of the blood clot happens due to its natural process with only the addition of inert anionic surfaces (e.g., glass or silicates) to initiate coagulation.

As used herein, “cultured clot serum” refers to serum that is collected from a cultured clot. In an embodiment, the cultured clot is centrifuged to collect the serum supernatant free of any cells. The supernatant free of any cell works as a cultured clot serum.

As used herein, the expression “effective duration” of culturing blood clot refers to time required for production of sLox-1. The duration of culturing blood clot suitable for use in some embodiments of the present invention typically ranges from zero to 10 mins to 20 mins, about 0.5 hours to 4 hours to about 5 weeks; varying from 1 hour, 2 hours, 3 hours, 4 hours, 5 hours, 6 hours, 7 hours, 10 hours, 15 hours, 20 hours, 24 hours, 2 days, 3 days, 4 days, 5 days or more.

As used herein, the expression “anti-coagulant factor” refers to an agent or class of agents that prevents coagulation or clotting of blood. For example but not limited to agents which function by chelating calcium as known in the art. These anticoagulants function by combining with, precipitating and effectively removing calcium ions normally present in the blood. They therefore reduce the concentration of calcium ion in the blood below normal physiological levels. Generally, the anticoagulants which fall within this definition include the citrate anticoagulants, for example, acid citrate dextrose (ACD), citrate phosphate dextrose (CPD), and trisodium citrate (TSC). Ethylenediaminetetraacetic acid (EDTA) may also be used. CPD is the most preferred anticoagulant of the available citrates, as described in U.S. Pat. No. 4,359,463A, which is incorporated herein by reference in its entirety. In an embodiment, heparin is an anticoagulant. In an embodiment, sLox-1 is an anti-coagulant.

As used herein, the term “coagulant” or like refers to a substance capable of causing whole blood or a blood component (plasma, platelets) to form a clot.

In an embodiment, the present invention may preferably use calcium ions to reverse the action attic citrate anticoagulant present in plasma feedstock, as described in US20040120942A1, which is incorporated herein by reference in its entirety.

As used herein, the term “blood” used herein means whole blood including hemocytes (erythrocytes, leucocytes, platelets) and plasma (serum) that is a liquid component, and liquid containing at least one of these (for example, blood collected by apheresis). The term “blood” refers to “fresh blood” that is not coagulated. In some embodiments, fresh blood is also interchangeably referred as an anticoagulated blood.

As used herein, the term “serum” means a pale-yellow liquid obtained by allowing collected blood to stand, resulting in reduction of the fluidity, followed by separation from the red coagulated block (clot). As used herein, the term “autologous blood” refers to a patient's own blood. As used herein, the term “homologous blood” refers to that obtained from a blood donor other than the individual for whom the coagulant is prepared.

As used herein, the term, “coagulation” is also known as clotting, is the process by which blood changes from a liquid to a gel, forming a blood clot. The mechanism of coagulation involves activation, adhesion and aggregation of platelets, as well as deposition and maturation of fibrin.

As used herein, the term, “coagulation enhancing factor or coagulation enhancing material” or like are agents that stimulate the contact pathway of coagulation (membranes containing phosphatidylserine that activate Factor XII) or like agents that respond in a cascade to form fibrin strands, which strengthen the platelet plug. The platelet plug is also known as the hemostatic plug or platelet thrombus, is an aggregation of platelets formed during early stages of hemostasis in response to one or more injuries to blood vessel walls. After platelets are recruited and begin to accumulate around the breakage, their “sticky” nature allows them to adhere to each other. This forms a platelet plug, which prevents more blood from leaving the body as well as any outside contaminants from getting in. The plug provides a temporary blockage of the break in the vasculature. As such, platelet plug formation occurs after vasoconstriction of the blood vessels but before the creation of the fibrin mesh clot, which is the more permanent solution to the injury. The result of the platelet plug formation is the coagulation of blood. For example, but not limited to Tissue factor (TF), a transmembrane glycoprotein in combination of binding factor FVII/FVIIa, lipopolysaccharide (LPS), Cancer procoagulant, etc.

As used herein, the term, “inflammation enhancing material” or like refers to the material that either leads or increases the inflammation reaction in mammals known to a person skilled in the art. For example, but not limited to Bacterial pathogens such as Lipopolysaccharide, Vasoactive amines, eicosanoids, peptidoglycan, to viral particles and etc. The term “enhancing material” refers to any material that increases the level of sLox-1 in cultured blood clot. For example, but not limited to phorbol esters such as phorbol myristate acetate (PMA), etc.

As used herein, the term, “viability” of the cells refers to cells not undergoing necrosis or late apoptosis. According to some embodiments, the term “viable cells” refers to cells having an intact plasma membrane. Assays for determining cell viability are known in the art, such as using alamarblue metabolic dye which may be detected in the serum by fluorimetry, or propidium iodide (PI) or calcein AM staining which may be detected by epifluorescence microscopy of the clot or flow cytometry of single cells. Accordingly, according to some embodiments, viable cells are cells which metabolize calcein AM and do not show propidium iodide intake. Necrosis can be further identified, by using light, fluorescence or electron microscopy techniques, or via uptake of the dye trypan blue.

As used herein, the term, “necrosis” of the cells is another mode of cell death as known in the art. A “necrotizing cell disease” includes trauma, ischemia, stroke, myocardial infarction, carbon lethal toxin-induced septic shock, sepsis, LPS-induced cell death and HIV-induced T cell death leading to immunodeficiency, it refers to acute diseases not limited to the above. The term “necrotic cell disease” also refers to chronic neurodegenerative diseases (eg Parkinson's disease, Huntington's disease, amyotrophic lateral sclerosis, Alzheimer's disease, infectious encephalopathy, dementia such as HIV-related dementia). But is not limited.

As used herein, the term, “activating agents” or like is used to provide a surface for reaction. Preferably, the activating agent provides a negatively charged catalytic surface that simulates the contact pathway of coagulation, or by exposure of blood to collagen type I. The intrinsic clotting cascade pathway is initiated by a process called contact activation, a surface and charge dependent phenomenon centered on the activation of Factor XII. Factor XII is highly susceptible to proteolysis because it is bound to surfaces via a charge interaction. The Factor XII precursor has areas of net positive charge that can interact with surfaces with a net negative charge. This charge binding induces conformational changes that enhance the molecule's ability to undergo activation by plasma kallikrein and Factor HK. “Activating agent” may also include thrombin.

Materials commonly used for contact activation include borosilicate glass (i.e., hematology glass), silicates, diatomaceous earth, ceramics, ellagic acid and kaolin. Ion exchange resins may also be suitable. Ion exchange resins can provide three separate process' functions in one single material: anionic activation of plasma proteins, a source of calcium to neutralize citrate, and molecular exclusion absorbance to remove water and low molecular weight fluids, thus, concentrating the high molecular weight constituents of the final serum. It has been demonstrated that a borosilicate glass with an anionic surface charge has these preferred features and can be used as an effective activating agent, as described in US20040120942A1 which is incorporated herein by reference it its entirety.

As used herein, the term, “interleukins (IL)” refers to a group of cytokines with complex immunomodulatory functions, including cell proliferation, maturation, migration and adhesion. In an embodiment of the invention, interleukins include human interleukin. ILs known to a person skilled in the art is covered according to an embodiment of the invention.

As used herein, the term, “coagulopathy” (also called a bleeding disorder) is a condition in which the blood's ability to coagulate (form clots) is enhanced or impaired. This condition can cause a tendency toward microthrombus formation or platelet depletion followed by prolonged or excessive bleeding (bleeding diathesis), which may occur spontaneously or following an injury or medical and dental procedures. The condition could result from multiple pathological, inheritable, trauma-induced or transfusion induced conditions, causing hypercoagulable or hypocoagulable states that may endanger life. Coagulopathy also occurs following acute trauma and hemorrhage in patients. In an embodiment, coagulopathy refers to platelet level dropped below 80,000 per microliter.

Trauma-induced coagulopathy is often the underlying cause of uncontrolled internal bleeding and, according to some accounts, leads to up to a fivefold increase in patient mortality. In order to normalize blood coagulation condition, a hemostasis therapy is essential that includes transfusion of whole blood or tissue factor concentrates. An early detection of the coagulopathy in patients and monitoring of coagulation metrics during the hemostasis therapy to guide therapeutic endpoints is important.

As used herein, the term, “cardiomyopathy” refers to diseases of the heart muscle. These diseases have many causes, signs and symptoms, and treatments. In cardiomyopathy, the heart muscle becomes enlarged, thick, or rigid. In rare cases, the muscle tissue in the heart is replaced with scar tissue. As cardiomyopathy worsens, the heart becomes weaker. It is less able to pump blood through the body and maintain a normal electrical rhythm. This can lead to heart failure or irregular heartbeats called arrhythmias. In turn, heart failure can cause fluid to build up in the lungs, ankles, feet, legs, or abdomen. The weakening of the heart also can cause other complications, such as heart valve problems.

The main types of cardiomyopathies are dilated cardiomyopathy, hypertrophic cardiomyopathy, restrictive cardiomyopathy, and arrhythmogenic right ventricular dysplasia. Other types of cardiomyopathies sometimes are referred as “unclassified cardiomyopathy.” Cardiomyopathy can be acquired or inherited, with hypertrophic cardiomyopathy and arrhythmogenic right ventricular dysplasia substantially being inherited disorders. In some subjects, inherited cardiomyopathies are not evident until the occurrence of a catastrophic event (e.g., heart attack). Cardiomyopathy can be induced by other diseases or conditions, or by various toxins or drugs. For example, dilated cardiomyopathy can result from coronary heart disease, heart attack, high blood pressure, diabetes, thyroid disease, viral hepatitis, and HIV; infections, especially viral infections that inflame the heart muscle can result in cardiomyopathy; alcohol, especially in conjunction with a poor diet, etc.

As used herein, the term, “LOX-1” or “Lox-1” as used herein is a type II transmembrane cell surface receptor, lectin-like oxidized low density lipoprotein receptor 1, first identified in endothelial cells as one of the main receptors for oxidized-LDL (ox-LDL). Besides ox-LDL, this receptor has been shown to bind many different ligands including other modified lipoproteins, advanced glycosylation end products, aged red blood cells, apoptotic cells, bacteria and activated platelets. Interestingly LOX-1 has been involved in many different pathological conditions including atherogenesis, myocardial ischemia, hypertension, vascular diseases, stroke, and thrombosis¹¹.

Expression of LOX-1 can be induced by a wide array of stimuli including pro-inflamatory factor (TNF-α, IL-1 or IFN-γ), angiotensin II, endothelin-1, modified lipoproteins and free radicals¹². Engagement of LOX-1 can lead to induction of oxidative stress, apoptosis, endothelial dysfunction, fibrosis and inflammation through the activation of the NF-κB pathway. LOX-1 has also been described to play a role in tumorigenesis. Indeed, LOX-1 up-regulation has been observed during cellular transformation into cancer cell and can have a pro-oncogenic effect by activating the NF-κB pathway, by increasing DNA damage through increase ROS production and by promoting angiogenesis and cell dissemination. The nucleic, acid sequence for the gene encoding LOX-1 (gene name OLR1) can be found in databases such as NCBI, i.e., NCBI gene ID: 4973 or Gene sequence: Ensembl: ENSG00000173391. The LOX-1 protein sequence is found at Hugo Gene Nomenclature Committee 8133, Protein Sequence HPRD:04003.

The term LOX-1 can also represent die receptor protein in various species, and with conservative changes in the amino acid or encoding sequences, or with other naturally occurring modifications that may vary among species and between members of the same species, as well as naturally occurring mutations thereof.

The term “soluble LOX-1” or “sLox-1” means that a part of LOX-1 existing in the membrane (usually part of “extracellular domain”) is cleaved (dissociated) and released into the blood. Means (shed) part of LOX-1. In an embodiment, sLox-1 is shown in FIG. 1.

Production of sLox-1

In an embodiment, a method of generating, ex vivo production of soluble Lox-1 (sLox-1), comprising introducing a blood sample into a device; adding a coagulation enhancing material in the device before clotting of the blood sample into the device; incubating the device; forming a cultured blood clot in the device; and shedding of the sLox-1 outside the cultured blood clot into the device.

In an embodiment, the blood sample has an anti-coagulant less than 10 wt. %, 9 wt. %, 8 wt. %, 7 wt. %, 6 wt. %, 5 wt. %, 4 wt. %, 3 wt. %, 2 wt. %, 1 wt. %, 0.5 wt. %, 0.1 wt. % or less.

In an embodiment, the blood sample is free from an anti-coagulant factor such as but not limited to heparin. 11111 In an embodiment, the blood sample is an anti-coagulated blood sample (fresh blood).

In an embodiment, the blood sample may contain coagulating enhancing material to accelerate formation of the clot.

In an embodiment, the blood sample may contain an enhancing material to stimulate sLox-1 production, for example comprises a lipopolysaccharide (LPS), a drug, or blood transfusion products (platelets, liposomes or microparticles), etc.

In an embodiment, the clot is incubated at the temperature around 20° C., 25° C., 30° C., 35° C., 37° C., 40° C., 45° C., 50° C. In an embodiment, the clot is incubated inside the device for about 0.5 hour (hr), 1 hr, 1.5 hrs, 2 hrs, 2.5 hrs, 3 hrs, 3.5 hrs, 4 hrs, 5 hrs, 6 hrs, 7 hrs, 8 hrs, 10 hrs, 12 hrs, 24 hrs or more.

In an embodiment, addition of the enhancing material in the blood forms a treated cultured clot. The cultured clot has about 20%, 30%, 40%, 50%, 60%, 70%, 80%, 90% or more production of sLox-1 compared to an untreated cultured blood clot free of the coagulation enhancing material.

In an embodiment, addition of the enhancing material in the blood forms a treated cultured clot. The cultured clot has about 1×, 2×, 3×, 4×, 5×, 6×, 7×, 8×, 9×, 10× or more production of sLox-1 compared to an untreated cultured blood clot free of the coagulation enhancing material.

In an embodiment, the method is configured to shed sLox-1 more than a fresh blood clot.

In an embodiment, during clot culture, the white blood cells are induced to express OLR1, the mRNA encoding Lox-1, and to shed on average 0.6 ng/mL sLox-1 into the serum. This novel technology enables the investigation of the role of donor sex, age, ethnicity, blood type, and other demographics in mechanisms of Lox-1 induction and sLox-1 shedding.

In an embodiment, the cultured clot system can be a precision medicine tool for detecting the capacity to induce sLox-1 appearance in the serum.

In an embodiment, sLox-1 can be readily and reproducibly generated in a serum sample, by placing whole blood from a donor at 37° C. The high levels of sLox-1 produced after just 4 hours of culture at 37° C. suggests that this approach may be used to generate autologous sLox-1 for clinical use. This approach could also be scalable for larger blood volumes from any species, to produce large amounts of bioactive sLox-1.

In an embodiment, our data shows that the thrombus may be a highly important source of Lox-1 expression and sLox-1 shedding.

In an embodiment, the method is configured to shed 0.5 ng to 50 ng of the sLox-1 per ml of the blood sample.

In an embodiment, the cultured clot serum sLox-1 concentration compared to fresh blood plasma is from 0 pg/mL to 10 ng/mL to 20 ng/mL to 30 ng/mL to 40 ng/mL to 50 ng/mL to 70 ng/mL to 100 ng/mL. In an embodiment, the concentration of sLox-1 in the cultured clot serum compared to fresh blood plasma is about 2 times, 3 times, 4 times, 5 times, 7 times, 10 times, 20 times, 50 times or 100 times more.

Lox-1 binds to phosphatidyl serine and to activated platelets which expose phosphatidyl serine to trigger the contact pathway for thrombin generation. It is therefore feasible that sLox-1 binds to platelet phosphatidyl serine, to have a two-fold effect in muffling the contact pathway activation of thrombin and limiting the ability of platelet microparticles to bind to endothelial cells.

Given these collective data, we postulate that thrombus-induced Lox-1 expression shedding of sLox-1 may be a natural protective mechanism whereby sLox-1 binds platelet microparticles to protect endothelial cells from becoming damaged by these pro-thrombogenic vesicles, and maintain hemostasis after traumatic injury (FIG. 1D).

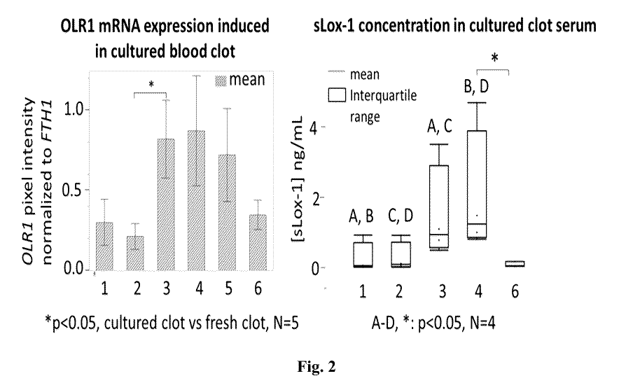

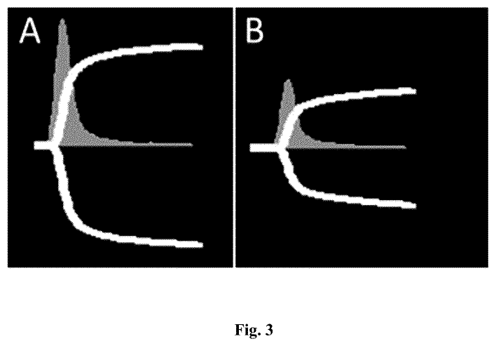

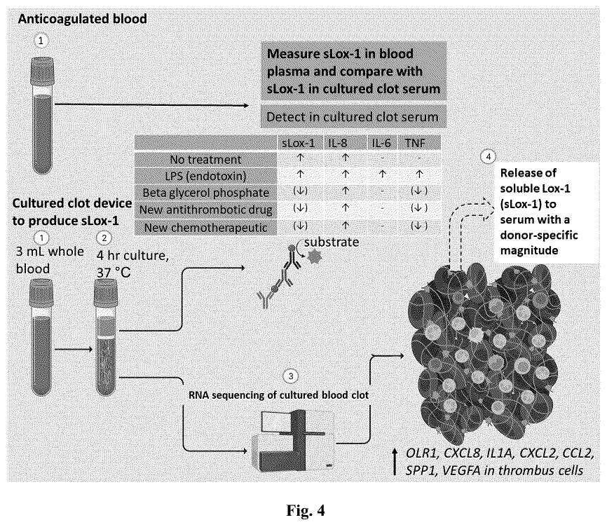

In an embodiment, cultured clots were spiked with LPS by injecting an LPS solution into the vacutainer tube immediately after blood collection and before clot formation. These LPS/cultured clots shed even more sLox-1 into the serum than the paired cultured clots without LPS, in line with the known effect of LPS and downstream mediators (TNF, IL1A) in stimulating Lox-1 expression. (FIG. 2).

In an embodiment, in the cultured clot serum and fresh blood, IL-8 and IL-6 concentrations are analyzed along with sLox-1 concentrations is also analyzed.

In an embodiment, the concentration of cultured clot serum IL-8 compared to fresh blood plasma is from 50 pg/mL to 10 ng/ml to 20 ng/ml to 40 ng/ml to 50 ng/ml to 70 ng/ml to 100 ng/ml.

In an embodiment, the concentration of cultured clot serum IL-8 compared to fresh blood plasma is about 2 times, 3 times, 4 times, 5 times, 7 times, 10 times, 20 times, 50 times or 100 times more.

In an embodiment, the concentration of IL-6 in cultured clot serum and/or fresh blood plasma is from 0 to 100 ng/mL. In an embodiment, the concentration of cultured clot serum IL-6 compared to fresh blood plasma is about 2 times, 3 times, 4 times, 5 times, 7 times, 10 times, 20 times, 50 times or 100 times more.

In an embodiment, the device may be used to screen blood donors for sLox-1 levels in order to produce lots of blood plasma or serum with “high” and “low” sLox-1 levels, for specific clinical applications where the level of sLox-1 could affect the clinical outcome, for example, inhibition of coagulation in organ transplantation, pro-coagulant activity for survival from hemorrhage.

In an embodiment, the method is configured to produce an autologous sLox-1.

### Diagnostic Application

In an embodiment, the cultured clot system is used as a novel diagnostic blood test that mimics a thrombus. The ex vivo thrombus device was optimized with blood samples from a cohort of 37 consenting healthy donors with diverse demographics (male/female, 18-61 years old, smoking or non-smoking; Caucasian/Hispanic/Latino/Asian/African-American; blood types ABO, Rh+/−, Lewis a/b/null, Duffy a/b/null, secretor/non-secretor).

The RNAseq study revealed that OLR1, the mRNA encoding Lox-1, is significantly induced in the ex vivo thrombus relative to fresh blood and fresh blood clots (FIG. 1B), along with the gene signature for MDSCs (CXCL8, CXCL2, CCL2, IL1A, TNF, VEGFA). Cultured clot histology showed Lox-1-expressing clot neutrophils and platelets.

Among 21 different healthy donors, 4 donors under 30 years old showed unexpectedly high baseline plasma sLox-1 levels (1.1-1.9 ng/mL). In addition, all healthy donors showed a personalized level of clot-induced sLox-1 shedding into cultured clot serum that was reproducible in blood draws from the same donors on different occasions.

Table 1 provides Donor Demographics for RNA sequencing study.

TABLE 1
Baseline characteristics in healthy blood donors (N = 10)

| | |
|---|---|
| Feeling healthy | yes |
| Age, y | 30 (20-60) |
| Covid status | Negative PCR test (N = 10) |
| Sex | 4 Female, 6 Male |
| BMI, kg/m² | 24.5 (21.5-28.8) |
| WBC × 10⁹/L | 7.1 (4.8-13.2) |
| Ethnicity | Caucasian, Asian, Hispanic/Latin, African |
| | American |
| ABO blood types | A, B, O |
| Rh-factor | Rh-positive, Rh-negative |
| Duffy blood alleles | Null, Lea, Leb, Lea/Leb |
| Lewis blood alleles | Null, Fyb, Fya/Fyb, Fya (nonsecretor) |
| Alcohol consumption # | 0.6 (0-2) |
| Smoking * | 0.5 (0-2) |
| Allergies (0 = none, 1 = yes) | 0.3 (0-1) |
| Pain medication | None |

BMI indicates body mass index; WBC, white blood cell count; # 0 = none, 1 = 1-3/week, 2 = 3+ per week; * 0 = never, 1 = former, 2 = yes.

In an embodiment, sLox-1 becomes elevated in blood plasma during thrombogenic viral infection.

In an embodiment, sLox-1 is a biomarker.

In an embodiment, present invention helps to detect cell apoptosis, scramblase activity, flippase activity, ADAM17 activity, ADAM10 activity, alpha secretase activity, sLox-1 sheddase activity, tumor necrosis factor activation.

In an embodiment, our novel data show that release of sLox-1 from the cultured clot is associated with those cells (neutrophils) that have a profile of PMN-MDSCs, which are known to drive cancer progression and inhibit immune defense against infection.

The term “biomarker” as described in this specification includes any physiological molecular form, or modified physiological molecular form, isoform, pro-form, naturally occurring forms or naturally occurring mutated forms of sLOX-1, expressed on the cell surface, unless otherwise specified. Other biomarkers that may be useful to detect cell apoptosis, scramblase activity, flippase activity, ADAM17 activity, ADAM10 activity, alpha secretase activity, sLox-1 sheddase activity, tumor necrosis factor activation It is understood that all molecular forms useful in this context are physiological, e.g., naturally occurring in the species. Preferably the peptide fragments obtained from the biomarkers are unique sequences. However, it is understood that other unique fragments may be obtained readily by one of skill in the art in view of the teachings provided herein.

By “isoform” or “multiple molecular form” is meant an alternative expression product or variant of a single gene in a given species, including forms generated by alternative splicing, single nucleotide polymorphisms, alternative promoter usage, alternative translation initiation small genetic differences between alleles of the same gene, and posttranslational modifications (PTMs) of these sequences.

“Gene Ontology” pathways induced in the cultured clot samples from 10 different (diverse) human donors that are induced by factors that are released from platelets (Transforming Growth Factor Beta). We also see pathways induced in the cultured clots from 5 different donors treated with lipopolysaccharide—that are activated by lipopolysaccharide, so the system is definitely working.

Table 2 shows GO pathways upregulated and downregulated in the cultured clotted blood.

In an embodiment, cultured clot device could be used to screen natural products derived from a host of microbial sources, yeast, bacteria, and screen for donor-specific inflammatory activity.

TABLE 2
GO pathways upregulated and downregulated in the cultured clotted blood samples.

| | GO Term; UPREGULATED: CULTURED CLOT VS | | | | | | |
|---|---|---|---|---|---|---|---|
| | ANTICOAGULATED BLOOD | Ont | N | Up | Down | P.Up | P.Down |
| GO:0072599 | SRP-dependent cotranslational protein targeting to | BP | 107 | 71 | 5 | 4.11418E−10 | 1 |
| | membrane | | | | | | |
| GO:0006613 | cotranslational protein targeting to membrane | BP | 94 | 64 | 3 | 6.95707E−10 | 1 |
| GO:0045047 | protein targeting to ER | BP | 104 | 68 | 5 | 2.7585E−09 | 1 |
| GO:0016070 | RNA metabolic process | BP | 2417 | 985 | 679 | 7.6843E−09 | 1 |
| GO:0032502 | developmental process | BP | 2593 | 1045 | 835 | 3.22322E−08 | 0.997810545 |
| GO:0022626 | cytosolic ribosome | CC | 91 | 59 | 7 | 5.28897E−08 | 1 |
| GO:0030154 | cell differentiation | BP | 1693 | 703 | 540 | 7.24897E−08 | 0.992740251 |
| GO:0009889 | regulation of biosynthetic process | BP | 2102 | 855 | 610 | 1.39456E−07 | 0.999999999 |
| GO:0048869 | cellular developmental process | BP | 1732 | 715 | 552 | 1.58995E−07 | 0.993869456 |
| GO:0070848 | response to growth factor | BP | 312 | 155 | 92 | 2.36224E−07 | 0.973011798 |
| GO:0001944 | vasculature development | BP | 313 | 155 | 93 | 2.8547E−07 | 0.966537216 |
| GO:0071363 | cellular response to growth factor stimulus | BP | 301 | 149 | 89 | 5.03828E−07 | 0.968574853 |
| GO:0009888 | tissue development | BP | 708 | 315 | 224 | 5.25253E−07 | 0.951952544 |
| GO:0072359 | circulatory system development | BP | 444 | 208 | 128 | 5.79882E−07 | 0.995232251 |
| GO:0048514 | blood vessel morphogenesis | BP | 258 | 130 | 80 | 7.64085E−07 | 0.888221411 |
| GO:0048856 | anatomical structure development | BP | 2381 | 956 | 772 | 8.68121E−07 | 0.991967192 |
| GO:0050793 | regulation of developmental process | BP | 1114 | 474 | 354 | 8.8679E−07 | 0.977298487 |
| GO:0007275 | multicellular organism development | BP | 2160 | 872 | 692 | 1.42986E−06 | 0.996508816 |
| GO:0006612 | protein targeting to membrane | BP | 154 | 85 | 24 | 1.81398E−06 | 0.999999978 |
| GO:1990830 | cellular response to leukemia inhibitory factor | BP | 45 | 31 | 8 | 7.99242E−06 | 0.995729675 |
| GO:0051171 | regulation of nitrogen compound metabolic process | BP | 2840 | 1117 | 867 | 1.27987E−05 | 0.999999963 |
| GO:0035295 | tube development | BP | 402 | 186 | 128 | 1.29135E−05 | 0.871345728 |
| GO:0004843 | thiol-dependent ubiquitin-specific protease activity | MF | 58 | 37 | 9 | 1.60005E−05 | 0.999614433 |
| GO:0001525 | angiogenesis | BP | 230 | 114 | 75 | 1.82277E−05 | 0.736412159 |
| GO:0043484 | regulation of RNA splicing | BP | 114 | 62 | 23 | 5.37545E−05 | 0.99972374 |
| GO:0006807 | nitrogen compound metabolic process | BP | 4813 | 1828 | 1540 | 6.99652E−05 | 0.999999981 |
| GO:0043066 | negative regulation of apoptotic process | BP | 455 | 204 | 132 | 8.17019E−05 | 0.99539686 |
| GO:0030509 | BMP signaling pathway | BP | 49 | 31 | 7 | 9.50506E−05 | 0.999558086 |
| GO:0071559 | response to transforming growth factor beta | BP | 112 | 59 | 32 | 0.000243179 | 0.918663089 |
| | Go Term: DOWNREGULATED CULTURED CLOT | | | | | | |
| | VS ANTICOAGULATED BLOOD | Ont | N | Up | Down | P.Up | P.Down |
| GO:0002275 | myeloid cell activation involved in immune response | BP | 453 | 108 | 227 | 0.999999999 | 9.15019E−13 |
| GO:0043299 | leukocyte degranulation | BP | 446 | 106 | 224 | 0.999999999 | 9.78558E−13 |
| GO:0002444 | myeloid leukocyte mediated immunity | BP | 458 | 112 | 227 | 0.999999994 | 3.63867E−12 |
| GO:0036230 | granulocyte activation | BP | 429 | 103 | 215 | 0.999999995 | 3.69916E−12 |
| GO:0045055 | regulated exocytosis | BP | 550 | 140 | 264 | 0.999999995 | 4.95593E−12 |
| GO:0042119 | neutrophil activation | BP | 424 | 101 | 212 | 0.999999996 | 6.8324E−12 |
| GO:0002443 | leukocyte mediated immunity | BP | 608 | 162 | 287 | 0.999999966 | 7.24162E−12 |
| GO:0071944 | cell periphery | CC | 2172 | 776 | 870 | 0.747102008 | 2.02024E−11 |
| GO:0002283 | neutrophil activation involved in immune response | BP | 417 | 99 | 207 | 0.999999996 | 2.78222E−11 |
| GO:0043312 | neutrophil degranulation | BP | 415 | 99 | 206 | 0.999999994 | 3.14096E−11 |
| GO:0002446 | neutrophil mediated immunity | BP | 424 | 102 | 209 | 0.999999993 | 4.99264E−11 |
| GO:0006955 | immune response | BP | 1349 | 422 | 565 | 0.999993741 | 1.43594E−10 |
| GO:0002366 | leukocyte activation involved in immune response | BP | 570 | 159 | 266 | 0.999996906 | 1.61592E−10 |
| GO:0005886 | plasma membrane | CC | 2049 | 731 | 819 | 0.756401928 | 1.73932E−10 |
| GO:0002263 | cell activation involved in immune response | BP | 573 | 159 | 267 | 0.999997984 | 1.78465E−10 |
| GO:0006887 | exocytosis | BP | 622 | 166 | 286 | 0.999999967 | 2.10103E−10 |
| GO:0005766 | primary lysosome | CC | 134 | 22 | 81 | 0.999999927 | 6.02838E−10 |
| GO:0031982 | vesicle | CC | 2142 | 714 | 847 | 0.999725127 | 1.72896E−09 |
| GO:0034774 | secretory granule lumen | CC | 234 | 50 | 124 | 0.99999988 | 2.30658E−09 |
| GO:0002252 | immune effector process | BP | 897 | 259 | 387 | 0.999999825 | 2.86053E−09 |
| GO:0035578 | azurophil granule lumen | CC | 75 | 7 | 51 | 0.999999988 | 3.41284E−09 |
| GO:0043230 | extracellular organelle | CC | 1189 | 389 | 494 | 0.998446117 | 1.05717E−08 |
| GO:0099503 | secretory vesicle | CC | 594 | 168 | 267 | 0.99999468 | 1.19739E−08 |
| GO:0045321 | leukocyte activation | BP | 911 | 293 | 388 | 0.998002764 | 1.48654E−08 |
| GO:1903561 | extracellular vesicle | CC | 1187 | 389 | 492 | 0.998192204 | 1.62932E−08 |
| GO:0001775 | cell activation | BP | 986 | 319 | 416 | 0.997943178 | 1.63864E−08 |
| GO:0070062 | extracellular exosome | CC | 1181 | 388 | 489 | 0.997735197 | 2.13583E−08 |
| GO:0016491 | oxidoreductase activity | MF | 341 | 91 | 162 | 0.999968748 | 1.65727E−07 |
| GO:0005759 | mitochondrial matrix | CC | 242 | 52 | 121 | 0.999999907 | 2.25406E−07 |
| | GO Term: UPREGULATED LPS/cultured clot vs | | | | | | |
| | cultured clot | Ont | N | Up | Down | P.Up | P.Down |
| GO:0006952 | defense response | BP | 952 | 353 | 185 | 1.33836E−43 | 0.999664101 |
| GO:0034097 | response to cytokine | BP | 747 | 298 | 122 | 2.85206E−43 | 0.999999924 |
| GO:0019221 | cytokinE−mediated signaling pathway | BP | 514 | 229 | 80 | 3.96671E−42 | 0.999998982 |
| GO:0043207 | response to external biotic stimulus | BP | 879 | 329 | 159 | 1.87029E−41 | 0.999990667 |
| GO:0051707 | response to other organism | BP | 879 | 329 | 159 | 1.87029E−41 | 0.999990667 |
| GO:0006955 | immune response | BP | 1349 | 432 | 316 | 1.61044E−35 | 0.615028294 |
| GO:0045087 | innate immune response | BP | 582 | 223 | 102 | 3.07093E−29 | 0.999921308 |
| GO:0034341 | response to interferon-gamma | BP | 140 | 83 | 14 | 4.08204E−26 | 0.999991177 |
| GO:0006950 | response to stress | BP | 2179 | 586 | 473 | 4.04312E−25 | 0.996182236 |
| GO:0009615 | response to virus | BP | 251 | 119 | 28 | 7.02835E−25 | 0.999999908 |
| GO:0006954 | inflammatory response | BP | 405 | 165 | 92 | 7.13008E−25 | 0.708216575 |
| GO:0044419 | biological process involved in interspecies interaction | BP | 1398 | 409 | 232 | 3.15439E−24 | 1 |
| | between organisms | | | | | | |
| GO:0071346 | cellular response to interferon-gamma | BP | 125 | 74 | 13 | 2.36E−23 | 0.999958163 |
| GO:0031347 | regulation of defense response | BP | 414 | 163 | 68 | 9.83303E−23 | 0.999921368 |
| GO:0001816 | cytokine production | BP | 486 | 183 | 105 | 1.09268E−22 | 0.890051916 |
| GO:0009617 | response to bacterium | BP | 339 | 138 | 70 | 4.00329E−21 | 0.916260383 |
| GO:0002682 | regulation of immune system process | BP | 958 | 297 | 202 | 4.99431E−21 | 0.981901904 |
| GO:0032496 | response to lipopolysaccharide | BP | 204 | 93 | 42 | 2.8311E−18 | 0.87365883 |
| GO:0060333 | interferon-gamma-mediated signaling pathway | BP | 73 | 47 | 7 | 2.72406E−17 | 0.999482311 |
| GO:0060337 | type I interferon signaling pathway | BP | 72 | 46 | 6 | 1.02842E−16 | 0.999838461 |
| GO:0007154 | cell communication | BP | 2892 | 691 | 734 | 2.16664E−15 | 0.005293075 |
| GO:0071356 | cellular response to tumor necrosis factor | BP | 183 | 79 | 26 | 3.83497E−14 | 0.999522027 |
| GO:0045321 | leukocyte activation | BP | 911 | 261 | 239 | 1.06492E−13 | 0.03407465 |
| GO:0002250 | adaptive immune response | BP | 297 | 109 | 50 | 2.89242E−13 | 0.998536556 |
| GO:0071347 | cellular response to interleukin-1 | BP | 130 | 60 | 11 | 1.06612E−12 | 0.9999983 |
| GO:1901700 | response to oxygen-containing compound | BP | 830 | 236 | 203 | 4.16441E−12 | 0.313783542 |
| GO:0002521 | leukocyte differentiation | BP | 356 | 121 | 81 | 8.55489E−12 | 0.694659362 |
| GO:0007249 | I-kappaB kinase/NF-kappaB signaling | BP | 205 | 80 | 44 | 1.96079E−11 | 0.811252848 |
| | GO Term: DOWNREGULATED LPS/cultured clot | | | | | | |
| Column1 | vs cultured clot | Ont | N | Up | Down | P.Up | P.Down |
| GO:0031224 | intrinsic component of membrane | CC | 1865 | 405 | 570 | 0.001969453 | 4.87431E−15 |
| GO:0071944 | cell periphery | CC | 2172 | 493 | 637 | 1.36951E−06 | 4.92262E−13 |
| GO:0005886 | plasma membrane | CC | 2049 | 466 | 604 | 3.28892E−06 | 1.01892E−12 |
| GO:0044255 | cellular lipid metabolic process | BP | 493 | 77 | 178 | 0.989143497 | 9.76827E−11 |
| GO:0120025 | plasma membrane bounded cell projection | CC | 807 | 131 | 25 | 0.99141735 | 8.75343E−08 |
| GO:0005887 | integral component of plasma membrane | CC | 494 | 139 | 163 | 3.31905E−07 | 5.22635E−07 |
| GO:0034308 | primary alcohol metabolic process | BP | 32 | 0 | 21 | 1 | 7.64507E−07 |
| GO:0046486 | glycerolipid metabolic process | BP | 232 | 37 | 84 | 0.923538416 | 1.06083E−05 |
| GO:1901615 | organic hydroxy compound metabolic process | BP | 228 | 39 | 83 | 0.837424219 | 1.13031E−05 |
| GO:0004888 | transmembrane signaling receptor activity | MF | 276 | 92 | 97 | 1.14262E−08 | 1.29311E−05 |
| GO:0009395 | phospholipid catabolic process | BP | 22 | 4 | 15 | 0.649134878 | 1.45733E−05 |
| GO:0006650 | glycerophospholipid metabolic process | BP | 201 | 30 | 74 | 0.957863239 | 1.82699E−05 |
| GO:0006644 | phospholipid metabolic process | BP | 232 | 35 | 83 | 0.963227807 | 2.08884E−05 |
| GO:0016042 | lipid catabolic process | BP | 135 | 23 | 54 | 0.79167843 | 2.11898E−05 |
| GO:0071617 | lysophospholipid acyltransferase activity | MF | 16 | 0 | 12 | 1 | 2.38279E−05 |
| GO:0016411 | acylglycerol O-acyltransferase activity | MF | 16 | 0 | 12 | 1 | 2.40985E−05 |
| GO:0038023 | signaling receptor activity | MF | 374 | 114 | 123 | 5.77453E−08 | 3.74021E−05 |
| GO:0060089 | molecular transducer activity | MF | 374 | 114 | 123 | 5.77453E−08 | 3.74021E−05 |
| GO:0003779 | actin binding | MF | 204 | 26 | 73 | 0.994449835 | 4.36134E−05 |
| GO:0006066 | alcohol metabolic process | BP | 168 | 28 | 63 | 0.846045154 | 4.96764E−05 |
| GO:0098590 | plasma membrane region | CC | 409 | 75 | 131 | 0.716022802 | 5.83167E−05 |
| GO:0008610 | lipid biosynthetic process | BP | 355 | 62 | 116 | 0.844777382 | 7.38181E−05 |
| GO:0042572 | retinol metabolic process | BP | 13 | 0 | 10 | 1 | 0.000101286 |
| GO:0044242 | cellular lipid catabolic process | BP | 100 | 14 | 41 | 0.940628158 | 0.000112021 |
| GO:0034754 | cellular hormone metabolic process | BP | 36 | 4 | 19 | 0.940893065 | 0.000180965 |
| GO:0043299 | leukocyte degranulation | BP | 446 | 99 | 138 | 0.06408957 | 0.000213609 |
| GO:0016788 | hydrolase activity, acting on ester bonds | MF | 354 | 60 | 113 | 0.890888128 | 0.000221491 |
| GO:0042171 | lysophosphatidic acid acyltransferase activity | MI | 14 | 0 | 10 | 1 | 0.000233947 |
| GO:0016298 | lipase activity | MF | 48 | 9 | 23 | 0.609295625 | 0.000249238 |

### Pharmaceutical Composition

In an embodiment, a precision medicine tool is developed that detects Lox-1 induction in an ex vivo thrombus and shedding of soluble Lox-1 (sLox-1) into the serum. The tool consists of a red vacutainer tube with as little as 0.5 mL and more ideally 3 or 4 mL of peripheral whole venous blood, that is placed for 4 hours at 37° C.

The data suggest that sLox-1 generated from a cultured clot may have cardioprotective activities. The cultured clot system may be used to create a sLox-1 based therapeutic that could prevent trauma-induced cardiomyopathy and diminish symptoms of acute coronary syndrome.

Data were generated that suggests cultured clot serum has an inhibitory effect on blood coagulation that may be due to accumulation of sLox-1 in the serum (FIG. 3). These data suggest that the cultured clot system may be used to generate sLox-1 for use a new biological anti-coagulant.

In an embodiment, sLox-1 may serve as a novel cardioprotective agent or new biological anti-coagulant.

In an embodiment, sLox-1 may serve as a novel anti-thrombogenic agent. Anti-thrombogenic refers to a process of delaying clotting of blood or delaying activation of pro-platelets or platelets in peripheral blood.

In an embodiment, anti-thrombogenic agent is served in an Anti-thrombogenic amounts. The term, “Anti-thrombogenic amounts”, without implying any limitation, “anti-thrombogenically effective” amount encompasses an amount that reduces some aspect of blood clotting to less than 100% of a maximal value, to less than 95% of a maximal value, to less than 90% of a maximal value, to less than 85% a maximal value, to less than 80%, to less than 75%, to less than 70%, to less than 65%, to less than 60%, to less than 55%, to less than 50%, to less than 40%, to less than 30%, to less than 20%, to less than 10%, to less than 5%, to less than 2%, to less than 1%, of the maximal value, and so on.

A “personalized pharmaceutical” shall mean specifically tailored therapies for one individual patient that will only be used for therapy in such individual patient, including actively personalized slox-1 using autologous patient's blood.

As used herein in its broadest meaning, the term “preventing” or “prevention” refers to preventing the disease or condition from occurring in a subject which has not yet been diagnosed as having it or which does not have any clinical symptoms.

As used herein, the term “treating” or “treatment”, as used herein, means reversing, alleviating, or inhibiting the progress of the disorder or condition to which such term applies, or one or more symptoms of such disorder or condition. A “therapeutically effective amount” is intended for a minimal amount of active agent which is necessary to impart therapeutic benefit to a subject. For example, a “therapeutically effective amount” to a patient is such an amount which induces, ameliorates, stabilises, slows down the progression or otherwise causes an improvement in the pathological symptoms, disease progression or physiological conditions associated with or resistance to succumbing to a disorder.

In an embodiment, sLox-1 could be used as a pharmaceutical composition.

As used herein, a “pharmaceutical composition” is a composition suitable for administration to a human being in a medical setting. Preferably, a pharmaceutical composition is sterile and produced according to GMP guidelines.

The pharmaceutical compositions comprise the protein extracted according to an embodiment of the invention, either in the free form or in the form of a pharmaceutically acceptable salt. As used herein, “a pharmaceutically acceptable salt” refers to a derivative of the protein extracted according to an embodiment of the invention, wherein the protein is modified by making acid or base salts of the agent. For example, acid salts are prepared from the free base (typically wherein the neutral form of the drug has a neutral —NH₂ group) involving reaction with a suitable acid. Suitable acids for preparing acid salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methane sulfonic acid, ethane sulfonic acid, p-toluene sulfonic acid, salicylic acid, and the like, as well as inorganic acids, e.g., hydrochloric acid, hydrobromic acid, sulfuric acid, nitric acid phosphoric acid and the like. Conversely, preparation of basic salts of acid moieties which may be present on a peptide are prepared using a pharmaceutically acceptable base such as sodium hydroxide, potassium hydroxide, ammonium hydroxide, calcium hydroxide, trimethylamine or the like.

In an embodiment, composition is used for parenteral administration, such as subcutaneous, intradermal, intramuscular or oral administration. For this, the proteins extracted according to an embodiment or other molecules are dissolved or suspended in a pharmaceutically acceptable, preferably aqueous carrier. In addition, the composition can contain excipients, such as buffers, binding agents, blasting agents, diluents, flavors, lubricants, etc. The proteins can also be administered together with immune stimulating substances, such as cytokines. An extensive listing of excipients that can be used in such a composition can be, for example, taken from A. Kibbe, Handbook of Pharmaceutical Excipients (Kibbe, 2000). The composition can be used for a prevention, prophylaxis and/or therapy of coagulopathy and/or cardiopathy.

In an embodiment, the pharmaceutical composition is administered in an therapeutically effective amount.

As used herein, the term “therapeutically effective amount” refers to an amount that is sufficient or effective to prevent or treat (delay or prevent the onset of, prevent the progression of, inhibit, decrease or reverse) a disease or condition described or contemplated herein, including alleviating symptoms of such disease or condition. As used herein, the term “effective amount” or “therapeutically effective amount” of a compound is that amount of compound that is sufficient to provide a beneficial effect to the subject to which the compound is administered. Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Device

In an embodiment, device may be used to screen blood donors for sLox-1 levels in order to produce lots of blood plasma or serum with “high” and “low” sLox-1 levels, for specific clinical applications where the level of sLox-1 could affect the clinical outcome, for example, inhibition of coagulation in organ transplantation, pro-coagulant activity for survival from hemorrhage, or to discriminate between low platelets arising from inadequate platelet production or platelet activation in the circulation which according to this invention is expected to stimulate sLox-1 shedding into the blood plasma.

In an embodiment, the device may be as shown in FIG. 28.

In an embodiment, cultured clot device to screen agents that promote or inhibit one or more of the following: cell apoptosis, scramblase activity, flippase activity, ADAM17 activity, ADAM10 activity, alpha secretase activity, sLox-1 sheddase activity, tumor necrosis factor activation.

In an embodiment, the device has a means of heating and a tube. The tube could be Vacutainer tube. The heating could be an inbuilt heating technique. The heating method could be portable.

In an embodiment, the device is capable to maintain the temperature at around 37° C. The temperature could be maintained for less than 1 hour, 2 hours, 3 hours, 4 hours, 5 hours, 6 hours, 7 hours, 8 hours, 24 hours, 48 hours or more.

In an embodiment, the device may have a cooling mechanism to decrease the temperature at a desired cooling temperature such as but not limited around 4° C. In an embodiment, the device is capable to decrease the temperature at around 4° C. automatically or manually after a specified time of usage for example: 1 hour, 2 hours, 3 hours, 4 hours, 5 hours, 6 hours, 7 hours, 8 hours, 24 hours, 48 hours or more.

FIG. 24 shows that the cultured clot device could be used as a precision medicine device to test for drug-induced responses that aim to reduce or increase the abundance of cells expressing Lox-1 (i.e., PMN-MDSCs), or alter sLox-1, or mediators upstream of sLox-1 shedding, including ADAM17 sheddase activity, phosphatidylserine exposure which is needed for ADAM17 activation, apoptosis which causes phosphatidylserine exposure, or TNF activity which depends on ADAM17.

In an embodiment, as shown in FIG. 28, Test article is a drug, biomaterial, pathogen, endotoxin, phorbol ester, phosphatase inhibitor or activator, kinase inhibitor or activator, antibody, nanoparticles, cells, mRNA, siRNA, CRISPR, cells, transfusion products, intended to modulate PMN-MDSC viability or activity, or oxidative stress, or ADAM17 activity or TNF activation or angiogenic activity.

In an embodiment, device could detect other soluble factors (i.e., high IL8 to document cell viability, no IL6 to document freedom from infection, TNF, IL1A, IL8, VEGF, resolvins, oxylipids, etc or other factors upregulated in cultured clots) for concentration and/or bioactivity.

In an embodiment, the device is as described in US20220133192A1.

### INCORPORATION BY REFERENCES

All references, including granted patents and patent application publications, referred herein are incorporated herein by reference in their entirety.

### Patent/Patent Application Publication

1. US20220133192A1
2. US20040120942A1
3. U.S. Pat. No. 4,359,463A Research Articles
(1) Sawamura, T.; Kume, N.; Aoyama, T.; Moriwaki, H.; Hoshikawa, H.; Aiba, Y.; Tanaka, T.; Miwa, S.; Katsura, Y.; Kita, T.; Masaki, T. An Endothelial Receptor for Oxidized Low-Density Lipoprotein. Nature 1997, 386 (6620), 73-77.
(2) Kume, N.; Kita, T. Lectin-Like Oxidized Low-Density Lipoprotein Receptor-1 (LOX-1) in Atherogenesis. Trends in Cardiovascular Medicine 2001, 11 (1), 22-25.
(3) Hofnagel, O.; Luechtenborg, B.; Stolle, K.; Lorkowski, S.; Eschert, H.; Plenz, G.; Robenek, H. Proinflammatory Cytokines Regulate LOX-1 Expression in Vascular Smooth Muscle Cells. Arteriosclerosis, Thrombosis, and Vascular Biology 2004, 24 (10), 1789-1795.
(4) Mehta, J. L.; Chen, J.; Hermonat, P. L.; Romeo, F.; Novelli, G. Lectin-like, Oxidized Low-Density Lipoprotein Receptor-1 (LOX-1): A Critical Player in the Development of Atherosclerosis and Related Disorders. Cardiovasc Res 2006, 69 (1), 36-45.
(5) Pirillo, A.; Norata, G. D.; Catapano, A. L. LOX-1, OxLDL, and Atherosclerosis. Mediators of Inflammation 2013, 2013, e152786.
(6) Akhmedov, A.; Sawamura, T.; Chen, C.-H.; Kraler, S.; Vdovenko, D.; Liischer, T. F. Lectin-like Oxidized Low-Density Lipoprotein Receptor-1 (LOX-1): A Crucial Driver of Atherosclerotic Cardiovascular Disease. Eur Heart J 2021, 42 (18), 1797-1807.
(7) Tatsuguchi, M.; Furutani, M.; Hinagata, J.; Tanaka, T.; Furutani, Y.; Imamura, S.; Kawana, M.; Masaki, T.; Kasanuki, H.; Sawamura, T.; Matsuoka, R. Oxidized LDL Receptor Gene (OLR1) Is Associated with the Risk of Myocardial Infarction. Biochemical and Biophysical Research Communications 2003, 303 (1), 247-250.
(8) Ryan, S.; Taylor, C. T.; McNicholas, W. T. Selective Activation of Inflammatory Pathways by Intermittent Hypoxia in Obstructive Sleep Apnea Syndrome. Circulation 2005, 112 (17), 2660-2667.
(9) Kent, B. D.; Ryan, S.; McNicholas, W. T. Obstructive Sleep Apnea and Inflammation: Relationship to Cardiovascular Co-Morbidity. Respiratory Physiology & Neurobiology 2011, 178 (3), 475-481.
(10) Chen, M.; Masaki, T.; Sawamura, T. LOX-1, the Receptor for Oxidized Low-Density Lipoprotein Identified from Endothelial Cells: Implications in Endothelial Dysfunction and Atherosclerosis. Pharmacology & Therapeutics 2002, 95 (1), 89-100.
(11) Weber, B.; Lackner, I.; Gebhard, F.; Miclau, T.; Kalbitz, M. Trauma, a Matter of the Heart—Molecular Mechanism of Post-Traumatic Cardiac Dysfunction. IDMS 2021, 22 (2), 737.
(12) Kakutani, M.; Masaki, T.; Sawamura, T. A Platelet—Endothelium Interaction Mediated by Lectin-like Oxidized Low-Density Lipoprotein Receptor-1. PNAS 2000, 97 (1), 360-364.
(13) Li, D.; Mehta, J. L. Antisense to LOX-1 Inhibits Oxidized LDL—Mediated Upregulation of Monocyte Chemoattractant Protein-1 and Monocyte Adhesion to Human Coronary Artery Endothelial Cells. Circulation 2000, 101 (25), 2889-2895.
(14) Mentrup, T.; Cabrera-Cabrera, F.; Schroder, B. Proteolytic Regulation of the Lectin-Like Oxidized Lipoprotein Receptor LOX-1. Front. Cardiovasc. Med. 2021, 7, 594441.
(15) Haywood-Watson, R. J.; Holcomb, J. B.; Gonzalez, E. A.; Peng, Z.; Pati, S.; Park, P. W.; Wang, W.; Zaske, A. M.; Menge, T.; Kozar, R. A. Modulation of Syndecan-1 Shedding after Hemorrhagic Shock and Resuscitation. PLoS ONE 2011, 6 (8), e23530.
(16) Kataoka, H.; Kume, N.; Miyamoto, S.; Minami, M.; Murase, T.; Sawamura, T.; Masaki, T.; Hashimoto, N.; Kita, T. Biosynthesis and Post-Translational Processing of Lectin-like Oxidized Low Density Lipoprotein Receptor-1 (LOX-1): N-LINKED GLYCOSYLATION AFFECTS CELL-SURFACE EXPRESSION AND LIGAND BINDING *. Journal of Biological Chemistry 2000, 275 (9), 6573-6579.
(17) Lee, A.-S.; Wang, Y.-C.; Chang, S.-S.; Lo, P.-H.; Chang, C.-M.; Lu, J.; Burns, A. R.; Chen, C.-H.; Kakino, A.; Sawamura, T.; Chang, K.-C. Detection of a High Ratio of Soluble to Membrane-Bound LOX-1 in Aspirated Coronary Thrombi From Patients With ST-Segment-Elevation Myocardial Infarction. Journal of the American Heart Association 2020, 9, e014008; 1-15.
(18) Hayashida, K.; Kume, N.; Murase, T.; Minami, M.; Nakagawa, D.; Inada, T.; Tanaka, M.; Ueda, A.; Kominami, G.; Kambara, H.; Kimura, T.; Kita, T. Serum Soluble Lectin-Like Oxidized Low-Density Lipoprotein Receptor-1 Levels Are Elevated in Acute Coronary Syndrome: A Novel Marker for Early Diagnosis. Circulation 2005, 112 (6), 812-818.
(19) Condamine, T.; Dominguez, G. A.; Youn, J.-I.; Kossenkov, A. V.; Mony, S.; Alicea-Torres, K.; Tcyganov, E.; Hashimoto, A.; Nefedova, Y.; Lin, C.; Partlova, S.; Garfall, A.; Vogl, D. T.; Xu, X.; Knight, S. C.; Malietzis, G.; Lee, G. H.; Eruslanov, E.; Albelda, S. M.; Wang, X.; Mehta, J. L.; Bewtra, M.; Rustgi, A.; Hockstein, N.; Witt, R.; Masters, G.; Nam, B.; Smirnov, D.; Sepulveda, M. A.; Gabrilovich, D. I. Lectin-Type Oxidized LDL Receptor-1 Distinguishes Population of Human Polymorphonuclear Myeloid-Derived Suppressor Cells in Cancer Patients. Sci Immunol 2016, 1 (2), aaf8943.
(20) Combadiere, B.; Adam, L.; Guillou, N.; Quentric, P.; Rosenbaum, P.; Dorgham, K.; Bonduelle, O.; Parizot, C.; Sauce, D.; Mayaux, J.; Luyt, C.-E.; Boissonnas, A.; Amoura, Z.; Pourcher, V.; Miyara, M.; Gorochov, G.; Guihot, A.; Combadiere, C. LOX-1-Expressing Immature Neutrophils Identify Critically-Ill COVID-19 Patients at Risk of Thrombotic Complications. Front. Immunol. 2021, 12, 752612.
(21) Ohno, M.; Kakino, A.; Sekiya, T.; Nomura, N.; Shingai, M.; Sawamura, T.; Kida, H. Critical Role of Oxidized LDL Receptor-1 in Intravascular Thrombosis in a Severe Influenza Mouse Model. Sci Rep 2021, 11, 15675.
(22) Veglia, F.; Sanseviero, E.; Gabrilovich, D. I. Myeloid-Derived Suppressor Cells in the Era of Increasing Myeloid Cell Diversity. Nat Rev Immunol 2021, 21 (8), 485-498.
(23) Hunyor, I.; Cook, K. M. Models of Intermittent Hypoxia and Obstructive Sleep Apnea: Molecular Pathways and Their Contribution to Cancer. American Journal of Physiology-Regulatory, Integrative and Comparative Physiology 2018, 315 (4), R669-R687.
(24) Leonard, J.; Lawson, M.; Gillevet, P.; Espina, V.; Hoemann, C. D. Hypoxic Whole Blood Clots Induce an Immune Response: A Potential Model for ARS and Covid-19; San Diego, 2020.
(25) Leonard, J. Creating an in Vitro Model for Acute Inflammation Using Tissue-Engineered Blood Clots. Masters thesis, George Mason University, Fairfax, V A, 2021.
(26) Aguillon, J. C.; Escobar, A.; Ferreira, V.; Aguirre, A.; Molina, M. C.; Ferreira, A. Daily Production of Human Tumor Necrosis Factor in Lipopolysaccharide (LPS)-Stimulated Ex Vivo Blood Culture Assays. European Cytokine Network 2001, 12 (1), 105-110.
(27) Moriwaki, H.; Kume, N.; Kataoka, H.; Murase, T.; Nishi, E.; Sawamura, T.; Masaki, T.; Kita, T. Expression of Lectin-like Oxidized Low Density Lipoprotein Receptor-1 in Human and Murine Macrophages: Upregulated Expression by TNF-α. FEBS Letters 1998, 440 (1), 29-32.
(28) Contreras-Garcia, A.; D'Elia, N. L.; Desgagné, M.; Lafantaisie-Favreau, C.-H.; Rivard, G.-E.; Ruiz, J.-C.; Wertheimer, M. R.; Messina, P.; Hoemann, C. D. Synthetic Anionic Surfaces Can Replace Microparticles in Stimulating Burst Coagulation of Blood Plasma. Colloids and Surfaces B: Biointerfaces 2019, 175, 596-605.
(29) del Conde, I.; Nabi, F.; Tonda, R.; Thiagaraj an, P.; López, J. A.; Kleiman, N. S. Effect of P-Selectin on Phosphatidylserine Exposure and Surface-Dependent Thrombin Generation on Monocytes. ATVB 2005, 25 (5), 1065-1070.
(30) Robilliard, L. D.; Kho, D. T.; Johnson, R. H.; Anchan, A.; O'Carroll, S. J.; Graham, E. S. The Importance of Multifrequency Impedance Sensing of Endothelial Barrier Formation Using ECIS Technology for the Generation of a Strong and Durable Paracellular Barrier. Biosensors 2018, 8 (3), 64.

1. A method of generating, ex vivo production of soluble Lox-1 (sLox-1), comprising:
introducing a sample containing blood into a device;
adding a coagulation enhancing material in the sample;
incubating a cultured blood clot in the device at a temperature greater than 25° C. and less than 45° C. for at least 2 hours
and
shedding of the sLox-1 outside the cultured blood clot, wherein the method is configured to shed sLox-1 more than an anti-coagulated blood.

2. The method of claim 1, wherein an additional enhancing material comprising a lipopolysaccharide (LPS) or phorbol myristate acetate and configured to modulate sLox-1 is added to the cultured blood clot.

3. The method of claim 1, wherein the anti-coagulant factor comprises heparin or citrate or ethylenediamine tetra acetic acid (EDTA).

4. The method of claim 1, wherein the cultured blood clot is configured to produce one or more interleukins.

5. The method of claim 4, wherein the one or more interleukins comprises IL-6, IL-8, IL-12, IL-36, IL-1A, IL-1B, and/or IL-18.

6. The method of claim 1, wherein addition of the coagulation enhancing material in the device spikes shedding of sLox-1 into the device by about 20% to 60% more compared to a cultured blood clot free of the coagulation enhancing material.

7. The method of claim 1, wherein the method is configured to shed 0.5 ng to 50 ng of the sLox-1 per ml of the blood sample.

8. The method of claim 1, wherein the method is configured to produce an autologous sLox-1.

9. The method of claim 1, the device is incubated at a temperature ranging from 20° C. to 45° C. for a time period ranging from at least 2 hour to 18 hours.

10. The method of claim 8, wherein the sLox-1 so sheds in the device is a personalized anti-coagulant agent.

11. The method of claim 1, wherein the device comprises a thrombus device.

12. The method of claim 1, wherein the method is configured to detect sLox-1, IL-8 and IL-6 concentrations in the cultured blood clot and the anti-coagulated blood.

13. The method of claim 1, wherein concentration of the sLox-1 in serum of the cultured clot compared to the anti-coagulated blood is from 0 pg/mL to about 50 ng/mL.

14. The method of claim 1, wherein concentration of the IL-8 in serum of the cultured clot compared to the anti-coagulated blood is from 50 pg/mL to 50 ng/ml.

15. The method of claim 1, wherein concentration of the IL-6 in serum of the cultured clot compared to the anti-coagulated blood is from 0 to 100 ng/mL.

16. A device comprising a vacutainer tube and a means of heating to maintain temperature of the device at about 25° C. to 45° C., wherein the device is configured to screen agents that promote or inhibit one or more of the following: cell apoptosis, scramblase activity, flippase activity, ADAM17 activity, ADAM10 activity, alpha secretase activity, sLox-1 sheddase activity, tumor necrosis factor activation.

17. The device of claim 16, wherein the device is configured to screen a drug, antibody, nanoparticle, nucleic acid, RNA-based nanoparticle, metal, vitamin, biomaterial, neutraceutical/dietary supplement as causing an increase or decrease in cultured clot serum sLox-1 relative to untreated cultured clot serum sLox-1, without inducing cell necrosis.

18. The device of claim 17, where the drug is a chemotherapeutic agent intended to induce apoptosis.

19. The device of claim 17, where the drug is configured to decrease ADAM17 activity and TNF activation.

20. The device of claim 17, where the drug is configured to suppress TNF expression or activity.

21. The device of claim 17, where the drug comprises a serine-threonine phosphatase inhibitor.

22. The device of claim 17, where the drug comprises beta glycerol phosphate.

23. The device of claim 1, where the device is used to screen for personalized responses to blood coagulation, inflammation-enhancing agents, drugs, proposed chemical treatments, or products derived from natural sources (bacteria, yeast, plants, cultured cells).

24. The device of claim 17, wherein the drug is configured to alter polymorphonuclear myeloid derived suppressor cell activity or viability.

25. The method of claim 1, wherein the blood is free of an anti-coagulant factor.
